(19) United States
(12) Patent Application Publication
(10) Pub. No.: US 2013/0204313 A1
(43) Pub. Date: Aug. 8, 2013

(54) SYSTEM TO DIAGNOSE THE FUNCTION OF INTRINSIC SPHINCTERS

(71) Applicant: PNEUMOFLEX SYSTEMS, LLC, Melbourne, FL (US)

(72) Inventors: W. Robert ADDINGTON, Melbourne Beach, FL (US); Stuart P. Miller, Indialantic, FL (US); Robert E. Stephens, Parkville, MO (US)

(73) Assignee: PNEUMOFLEX SYSTEMS, LLC, Melbourne, FL (US)

(21) Appl. No.: 13/838,196

(22) Filed: Mar. 15, 2013

Related U.S. Application Data
(63) Continuation-in-part of application No. 13/456,841, filed on Apr. 26, 2012.
(60) Provisional application No. 61/480,625, filed on Apr. 29, 2011.
(60) Provisional application No. 61/533,389, filed on Sep. 12, 2011.

(51) Int. Cl. A61N 1/36

(52) U.S. Cl. CPC A61N 1/36007
USPC 607/41

(57) ABSTRACT

A system treats at least one of the urethral and anal sphincters. A muscle contraction device is located on at least one of the urethral and anal sphincters. A controller is connected to the muscle contraction device and operative to transmit control signals to the muscle contraction device located on at least one of the urethral and anal sphincters to contract the muscle during an inspiratory phase of respiration.

### RELATED APPLICATION(S)

This application is a continuation-in-part application of commonly assigned U.S. patent application Ser. No. 13/456,841 filed on Apr. 26, 2012; and this application claims priority to U.S. provisional patent application Ser. No. 61/738,027, filed Dec. 17, 2012, and U.S. provisional patent application Ser. No. 61/756,246, filed Jan. 24, 2013, the disclosures which are hereby incorporated by reference in their entirety.

### FIELD OF THE INVENTION

The present invention relates to initiating an involuntary reflex cough test for diagnosing physiological abnormalities, and more particularly, to testing and diagnosing at least the urethral sphincter.

### BACKGROUND OF THE INVENTION

Commonly assigned U.S. patent application Ser. No. 13/456,841 discloses a system and method that tests the gastric valve and urethral sphincter in a patient. A contrast agent is administered into the esophagus of a patient followed by inducing an involuntary reflex cough epoch within the patient to isolate the gastric valve from the lower esophageal sphincter (LES) and isolate the external urethral sphincter from the internal urethral sphincter. An imaging sensor detects the flow of the contrast agent during the involuntary reflex cough epoch and determines whether stomach reflux occurred indicative of a malfunctioning gastric valve. A determination is made if urine leakage occurs indicative of stress urinary incontinence (SUI).

The flow of contrast agent can be detected at the level of the LES using a fluoroscopic instrument configured to image the contrast agent. A chemo-irritant can induce the involuntary reflex cough epoch using a nebulizer. Barium sulfate is a preferred contrast agent that is swallowed by the patient. Typically, the involuntary reflex cough epoch is induced following the administration of that contrast agent.

A urinary catheter having a pressure sensor is inserted within the bladder. An EMG is obtained from the involuntary cough activated intercostals and the data processed from the pressure sensor with the EMG to estimate the severity of the SUI. The EMG can be obtained from the paraspinals.

It is desirable if further analysis and treatment of the internal urethral sphincter (IUS) and internal anal sphincter (IAS) are accomplished by observing the pulmonary inspiration efferents that illicit a pattern reflex motor response to test and diagnose lower esophageal sphincter (LES) and/or the internal urethral sphincter (IUS).

### SUMMARY OF THE INVENTION

This summary is provided to introduce a selection of concepts that are further described below in the detailed description. This summary is not intended to identify key or essential features of the claimed subject matter, nor is it intended to be used as an aid in limiting the scope of the claimed subject matter.

A system treats at least one of the urethral and anal sphincters. A muscle contraction device is located on at least one of the urethral and anal sphincters. A controller is connected to the muscle contraction device and operative to transmit control signals to the muscle contraction device located on at least one of the urethral and anal sphincters to contract the muscle during an inspiratory phase of respiration.

Sensors are positioned to detect movement of the ribs indicative of the inspiratory phase of respiration. The sensors are positioned using sensors placed at the interior surface of the medial border of the costal margin of ribs 8, 9 or 10. A muscle contraction device is located on at least one of the internal and external urethral sphincters. The muscle contraction device includes an electrode to stimulate the muscle using an electrical current. A controller is operative to transmit control signals to the electrodes to initiate electrical stimulation in another example. A controller is embedded in the abdominal wall in an example and microwires connect the controller and electrodes. The muscle contraction device may be formed as a mechanical mechanism in another example. The muscle may be contracted during an inspiratory phase of respiration based on inspiration/expiration endpoints of rib cage movement.

### BRIEF DESCRIPTION OF THE DRAWINGS

Other objects, features and advantages of the present invention will become apparent from the detailed description of the invention which follows, when considered in light of the accompanying drawings in which:

FIG. 1 is an anterior view of the human torso showing sensors such as transducers positioned to detect movement of the ribs during inspiration and expiration in accordance with a non-limiting example.

FIG. 2 is an anterior view of the human thoracic skeleton showing the sensors such as transducers to detect movement of the ribs during inspiration and expiration in accordance with a non-limiting example.

FIG. 3 is a medial view of a female pelvis showing sensors as transducers, electrodes and a processor as part of a receiver in accordance with the non-limiting example.

FIG. 4 is a medial view of a female pelvis showing sensors and transducers to detect inspiration and simulate the electrodes and operating wirelessly in accordance with a non-limiting example.

FIG. 4A is a flowchart showing a basic sequence of operation.

FIG. 5A are images of the inspiration closure reflex (ICR) and showing the BFV sequences for barium swallow followed by deep inspiration that allows barium to enter the stomach.

FIG. 5B is a nerve conduction pathway circuit diagram for the inspiration closure reflex (ICR) showing how intrinsic sphincter tenacity is regulated during inspiration and expiration in accordance with a non-limiting example.

FIG. 6A are images showing a barium swallow during a breath-hold of a patient and depicting inspiration followed by barium swallow.

FIG. 6B is a nerve conduction pathway circuit diagram to show the physiology of the breath-hold as in FIG. 6A in accordance with a non-limiting example.

FIG. 7 are images showing the barium swallow during breath-hold in accordance with a non-limiting example.

FIG. 8A are images showing the laryngeal expiratory reflex which the LES appear patent during the LER cough epoch in accordance with a non-limiting example.

FIG. 8B shows a nerve conduction pathway circuit diagram of the stimulation of laryngeal receptors using the involuntary reflex cough test.

FIGS. 9A and 9B are graphs showing pressure recordings of the IUS and LES synchronizes with respiration in accordance with a non-limiting example.

FIG. 10 is a graph showing relative latencies of the IUS and LES with deep inspiration and expiration in accordance with a non-limiting example.

FIG. 11 is a graph showing the breath-hold with maintained pressure elevation in the LES and IUS in accordance with a non-limiting example.

FIGS. 12A and 12B are graphs showing the urodynamic tracing of a series of forceful voluntary coughs in accordance with a non-limiting example.

FIG. 13 is another nerve conduction pathway circuit diagram showing the inspiration closure reflex in accordance with a non-limiting example.

FIG. 14 is a flowchart illustrating a sequence of steps for isolating the gastric valve to assess its function in accordance with a non-limiting example.

FIG. 15 is another flowchart illustrating a sequence of steps for isolating the gastric valve and external urethral sphincter to assess their function in accordance with a non-limiting example.

FIG. 16A is a fragmentary view of an example of a kit having components for use with the methodology described relative to FIGS. 14 and 15 in accordance with a non-limiting example.

FIG. 16B is a view showing a system that includes a patient bed as a platform and imaging sensor for performing the methodology of FIGS. 14 and 15.

FIG. 17 is a simplified plan view of a catheter that can be used for urodynamic and medical diagnostic testing in accordance with a non-limiting example.

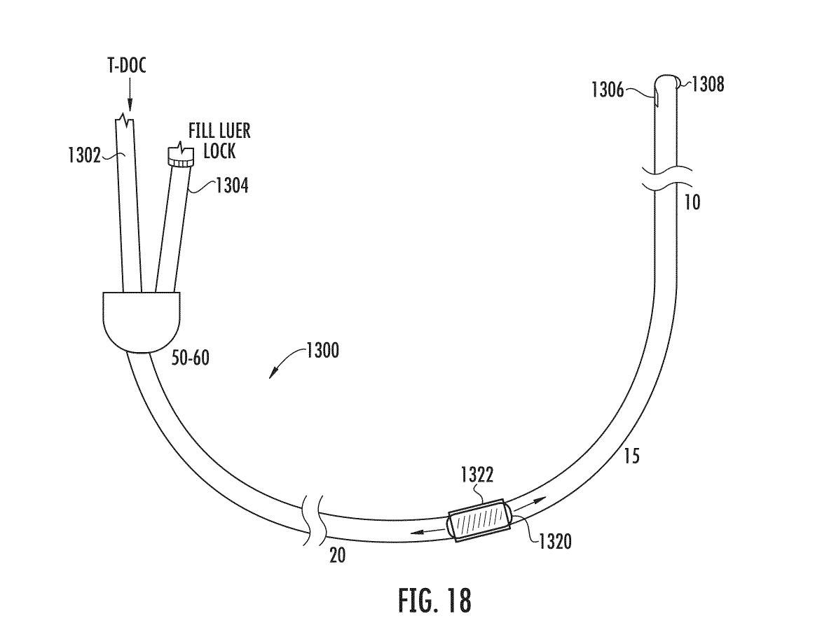

FIG. 18 is a simplified plan view of another example of a catheter similar to that shown in FIG. 17 that can be used for urodynamic and medical diagnostic testing in accordance with a non-limiting example.

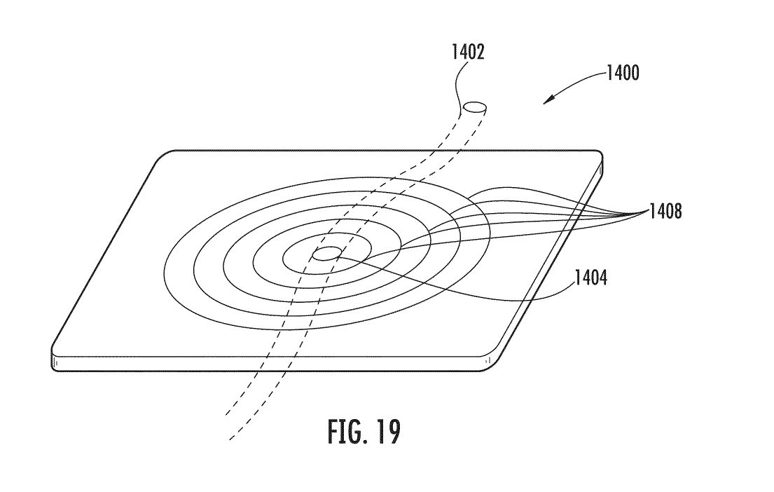

FIG. 19 shows a urinary continence pad that can be used with urodynamic catheters of FIGS. 17 and 18.

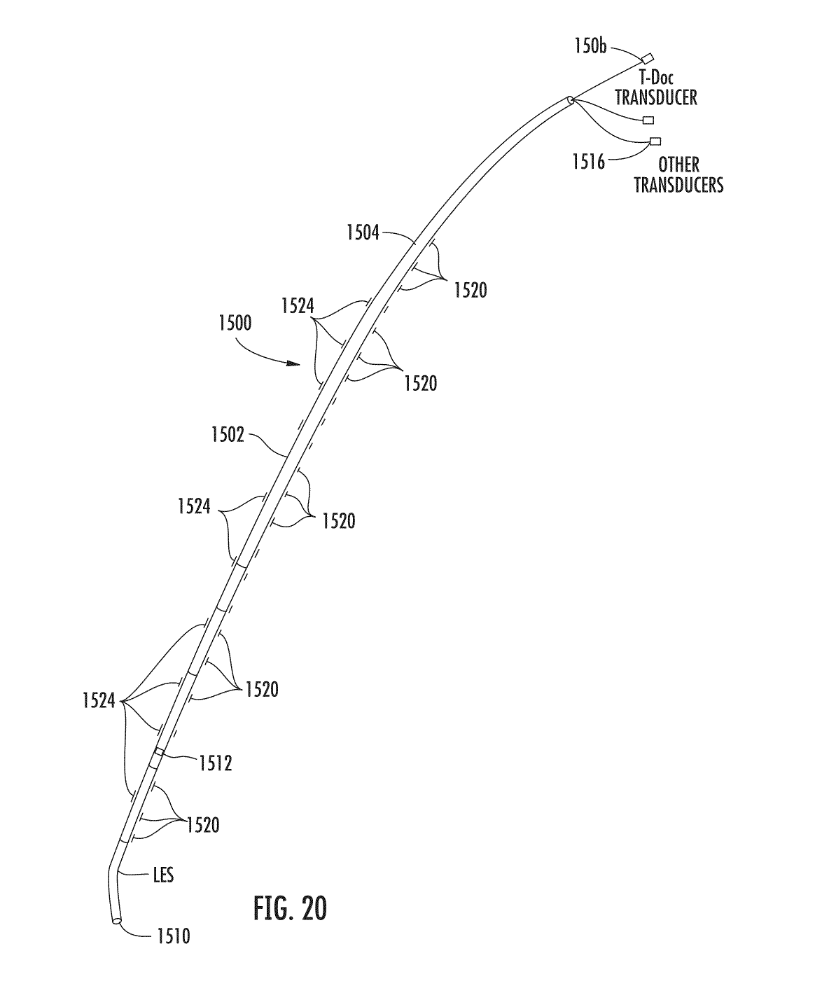

FIG. 20 is a plan view of an Ng/Og device or catheter that can be used for testing for acid reflux.

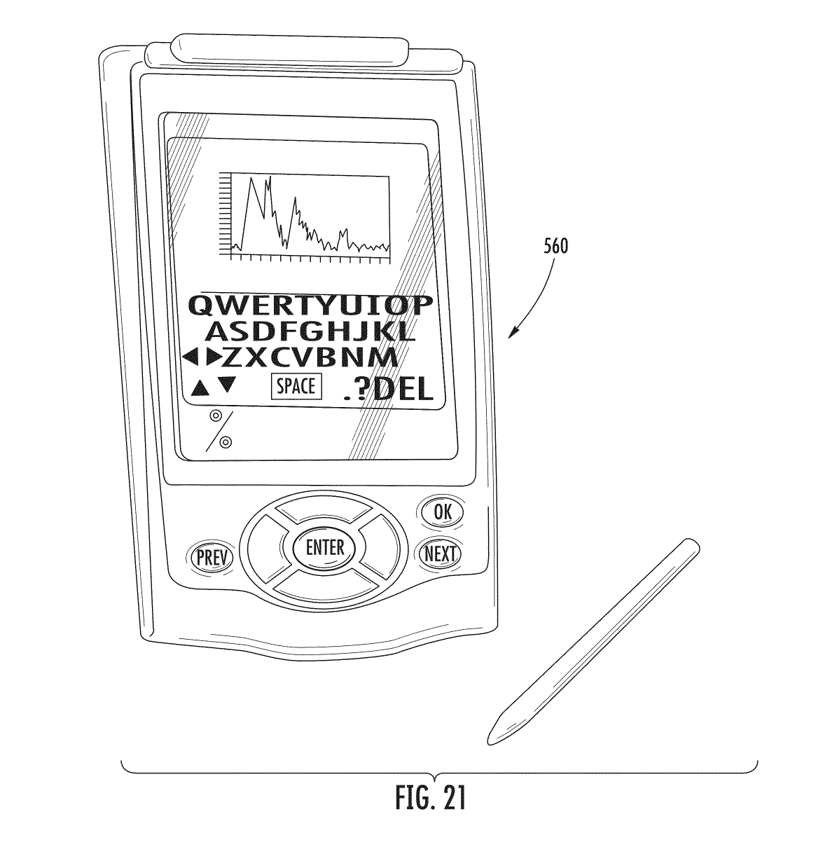

FIG. 21 is a fragmentary plan view of a handheld processing device that can be used in conjunction with various catheters and Ng/Og devices or other catheters and/or nebulizers.

FIG. 22 is a block diagram showing example components of a handheld processing device such as shown in FIG. 21.

FIG. 23 is a block diagram showing an outline of the laryngeal expiratory reflex (LER) and results with the intrinsic sphincter deficiency and esophageal, urinary and fecal continence.

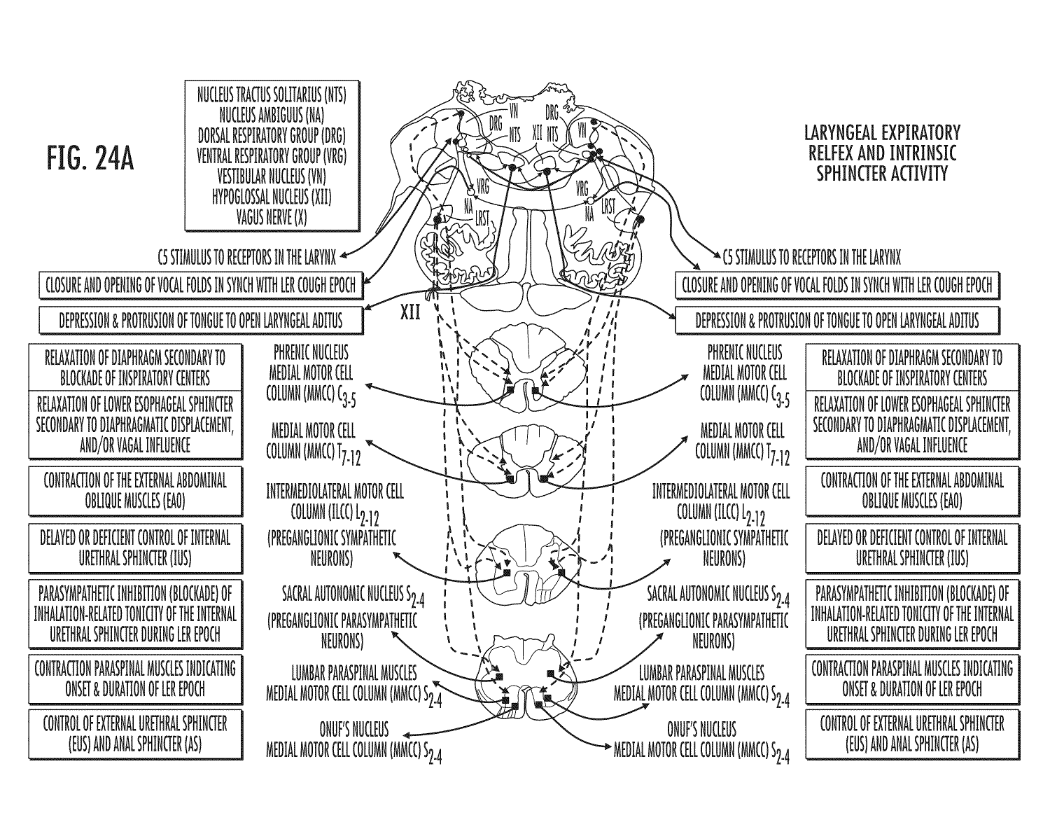

FIGS. 24A and 24B are graphs detailing what occurs during LER with intrinsic sphincter activity (FIG. 24A) and voluntary cough pathways (FIG. 24B).

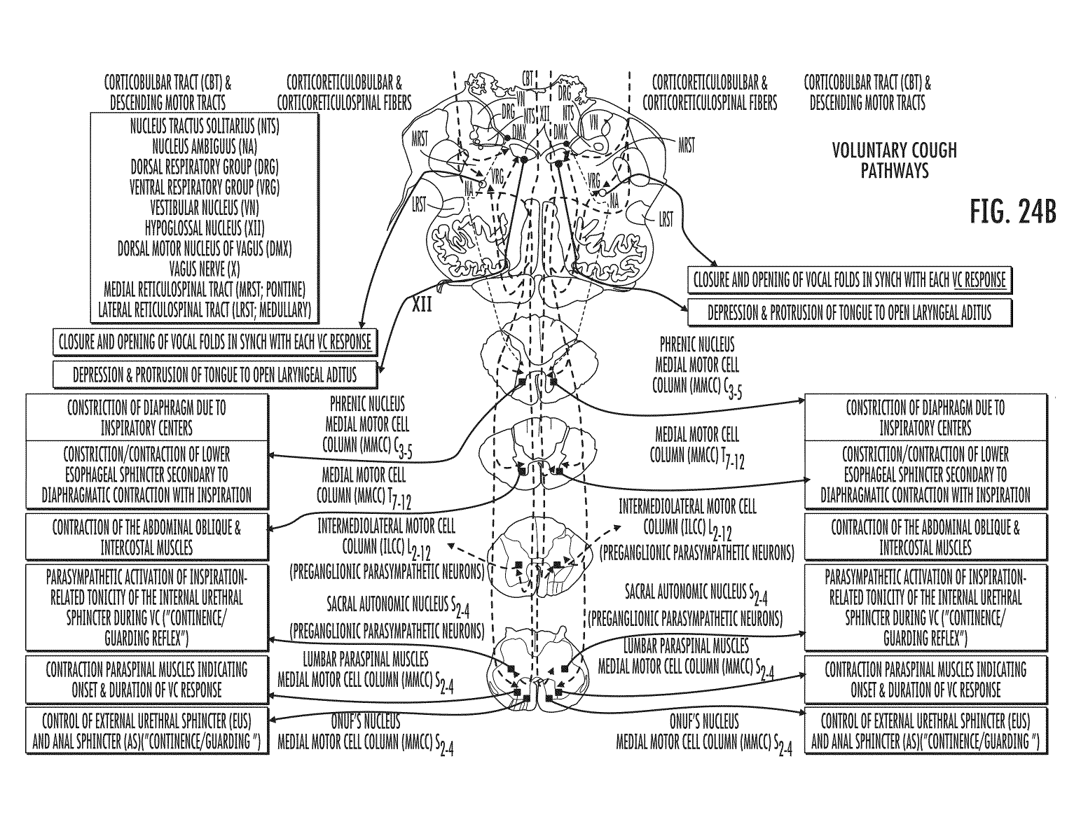

### DETAILED DESCRIPTION OF THE PREFERRED EMBODIMENTS

Different embodiments will now be described more fully hereinafter with reference to the accompanying drawings, in which preferred embodiments are shown. Many different forms can be set forth and described embodiments should not be construed as limited to the embodiments set forth herein. Rather, these embodiments are provided so that this disclosure will be thorough and complete, and will fully convey the scope to those skilled in the art.

Commonly assigned U.S. patent application Ser. Nos. 13/456,882 and 13/456,841 filed on Apr. 26, 2012, (U.S. Patent Publication Nos. 2012/0277583 and 2012/0277547) by the same inventors and which are hereby incorporated by reference in their entirety, disclose system and methods for testing the gastric valve and urethral sphincter and with analysis of the lower esophageal sphincter. Further developments, however, have now been made at observing the effect of respiration on intrinsic sphincters such as the lower esophageal sphincter (LES) and internal urethral sphincter (IUS).

The functions of the lower esophageal sphincter (LES) an internal urethral sphincter (IUS) have now been analyzed during voluntary and involuntary respiratory maneuvers. A prospective barium videoflouroscopy study (BSV) of the LES on four healthy adult men during voluntary cough (VC) was performed together with the laryngeal expiration reflex (LER), breath-hold maneuvers, and normal inspiration. One subject had fiber-optic pressure catheters placed in the LES and IUS, and electromyographic recording of the right T7-8 intercostals during respiration. The BSV showed closure and relaxation of the LES corresponding to the inspiration and expiration of VC. The LES was patent during the LER. There was closure of the LES during the deep inspiration/breath-hold event. Pressure catheters in the LES and IUS showed increased pressure during inspiration. These observations suggest that pulmonary inspiration afferents elicit a patterned reflex motor response in the LES and IUS, referred to as the inspiration closure reflex (ICR).

Test results have determined there is an Inspiration Closure Reflex (ICR) control of the IUS (Internal Urethral Sphincter). An IAP (Intra abdominal Pressure) transducer has been used for the study and data. A processor is programmed to correlate the IA (Intra abdominal) pressure changes and the associated duration of each event (as detected by the pressure transducer) with corresponding stimulation of the smooth muscle of the IUS, and/or the striated muscle of the EUS (external urethral sphincter) and/or AS (anal sphincter). Muscle stimulators may be implanted using trans-urethral or trans-vaginal approaches and connected to a processor as part of a receiver either directly such as with microwires or indirectly such as using wireless communication, for example, Bluetooth or other wireless communication.

A trans-vaginal approach is favored when a microwire is inserted via subcutaneous trochar as from the mid-line, suprapubic and inferior border of the pubic ramus. Vaginal palpation occurs at the distal end of the microwire for a muscle stimulator to the IUS, EUS, and/or AS. The confirmation of placement may be accomplished by the use of a urethral pressure catheter. It is possible to palpate the wire passing posterior to the vagina and palpate its placement adjacent to the AS surrounding the anus as distal about one inch of the rectum. The AS stimulation is confirmed by a rectal pressure catheter. The transducer and microprocessor may be connected to the stimulator via a wireless connector. A power source and electronic stimulator may be proximate to the targeted sphincter muscles in this system and apparatus.

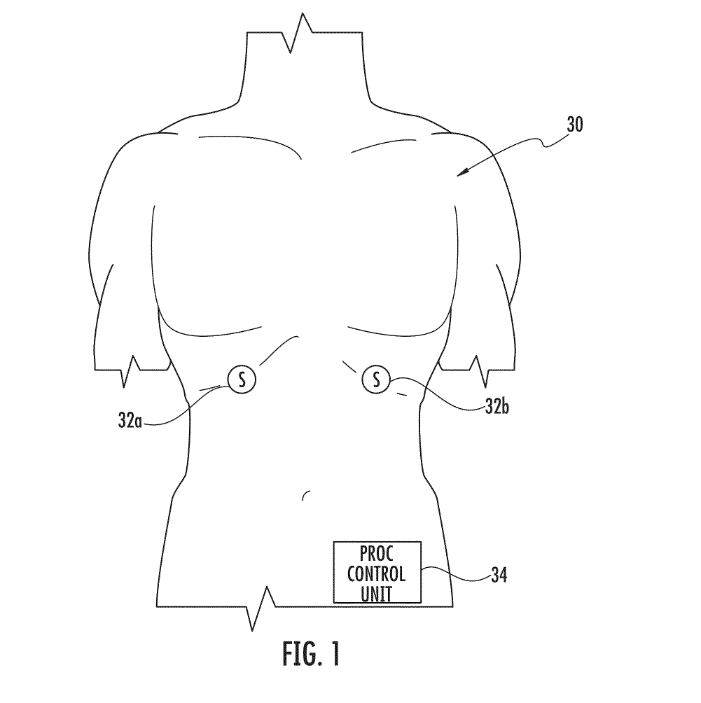

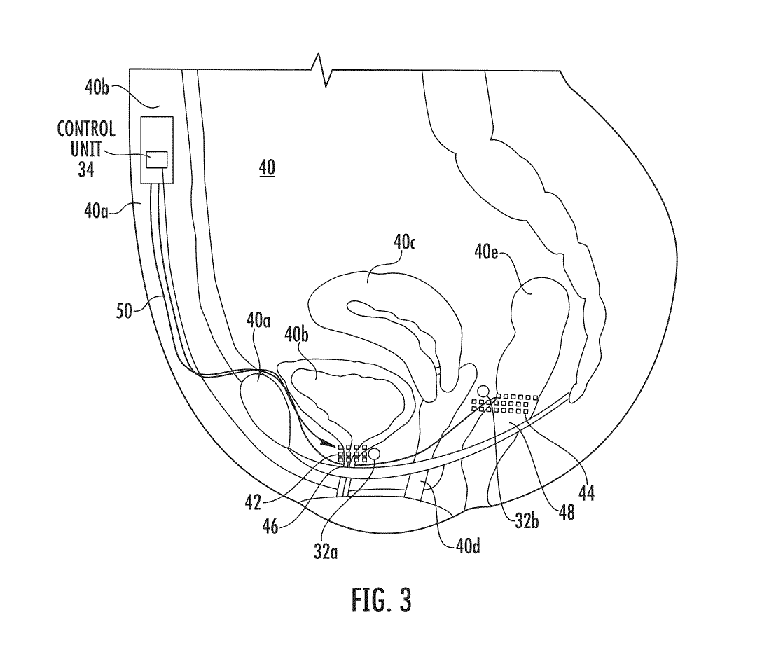

FIG. 1 shows an anterior view of the human torso at 30. The sensors 32a, 32b as transducers in this example are positioned to detect movements of the ribs during inspiration and expiration and are placed at the anterior surface of the medial border of the costal margin of ribs 8, 9 or 10. The sensors 32a, 32b in this example are formed as transducers to measure movement. These sensors 32a, 32b (either on ore two) are directly or indirectly connected to a control unit 34 operative as a controller having a signal receiver/transceiver, which in this example is embedded in the subcutaneous fat of the lower quadrant of the abdominal wall. The control unit 34 controls the electrodes 42, 44 (FIG. 3) that simulate contraction of the internal urethral sphincter (IUS) and internal anal sphincter during the inspiratory phase of respiration.

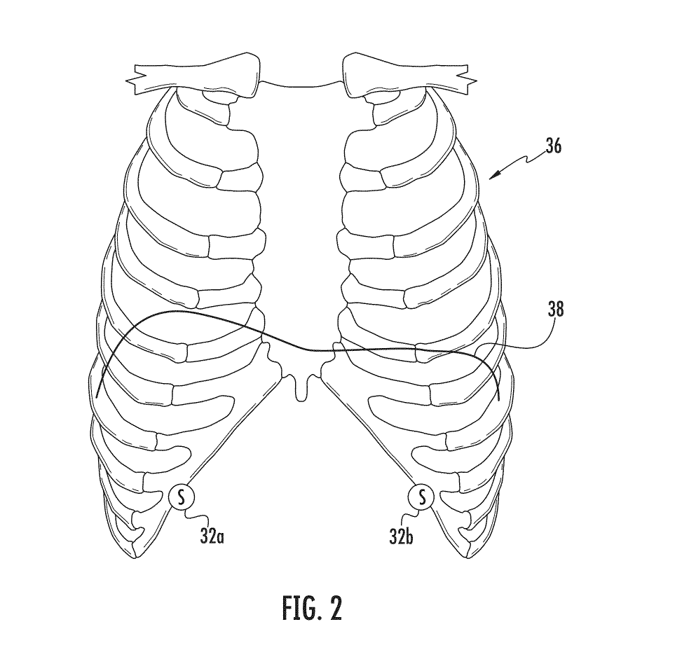

FIG. 2 shows an anterior view of the human thoracic skeleton 36. The osteocartilaginous thoracic cage 36 as illustrated includes the sternum, 12 pairs of ribs and associated costal cartilages and 12 thoracic vertebrae and intervertebral discs. The position of the superior domes of the diaphragm is indicated by the line 38. The sensors 32a, 32b are configured to detect movement of the ribs during inspiration and expiration and are placed on the anterior surface of the medial border of the costal margin of ribs 8, 9 or 10 in this example. The sensors 32a, 32b may be placed either unilaterally or bilaterally.

FIG. 3 is a medial view of the female pelvis 40. A microprocessor is part of a control unit 34 and implanted in the fatty layer (Camper's fascia 40a) or in the deep layer (Scarpa's fascia 40b) and against the fascia of the external abdominal oblique muscle in the patient's left or right lower abdominal wall. This control unit 34 receives wireless signals from the sensors 32a, 32b regarding movement of the lower rib cage during inspiration. The control unit 34 will detect the onset of inspiration and simulate microelectrodes 42, 44 that are implanted in the respective internal urethral sphincter (IUS) 46 and internal anal sphincter (IAS) 48 indicated by the multiple dotted lines on the appropriate sphincter. The control unit 34 is connected to the microelectrodes 42, 44 (single or as an array of microelectrodes either directly (via microwire) or indirectly (wireless, radio frequency, Blue-Ray, or similar technology). These pelvic devices as the microelectronics may close the IUS or IAS either electronically (muscle simulation) or activate mechanical mechanisms that close the IUS and IAS with or without an intervening processor. Microwires 50 if used are placed using a trochar to tunnel through Camper's fascia 40a (subcutaneous fat of the abdominal wall) to the superior border of the pubic bone. There is also illustrated the pubis 40a, urinary bladder 40b, uterus 40c, vagina 40d, and rectum 40e.

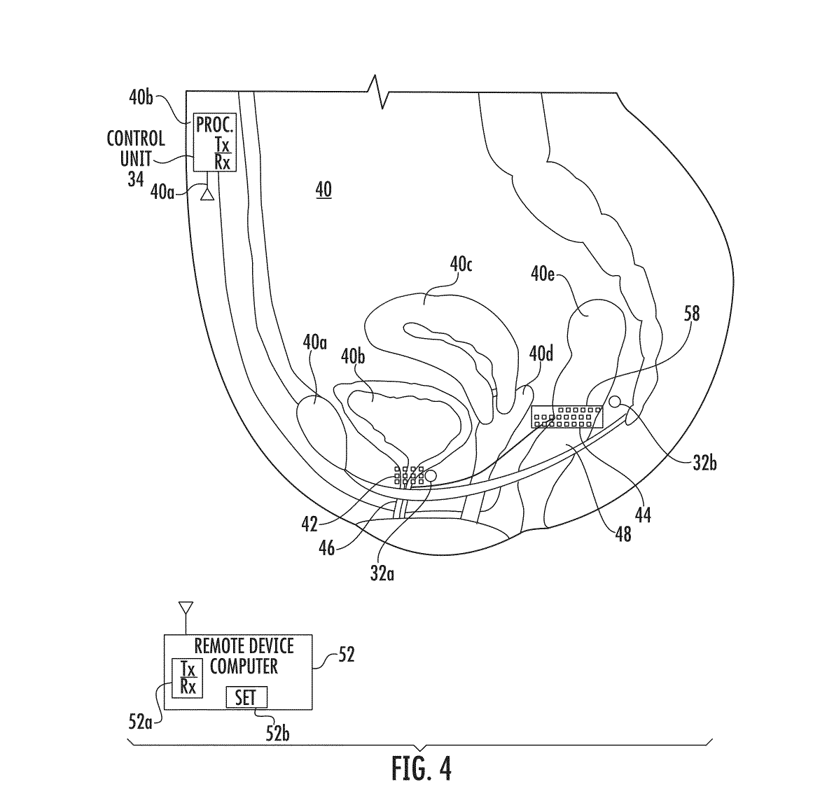

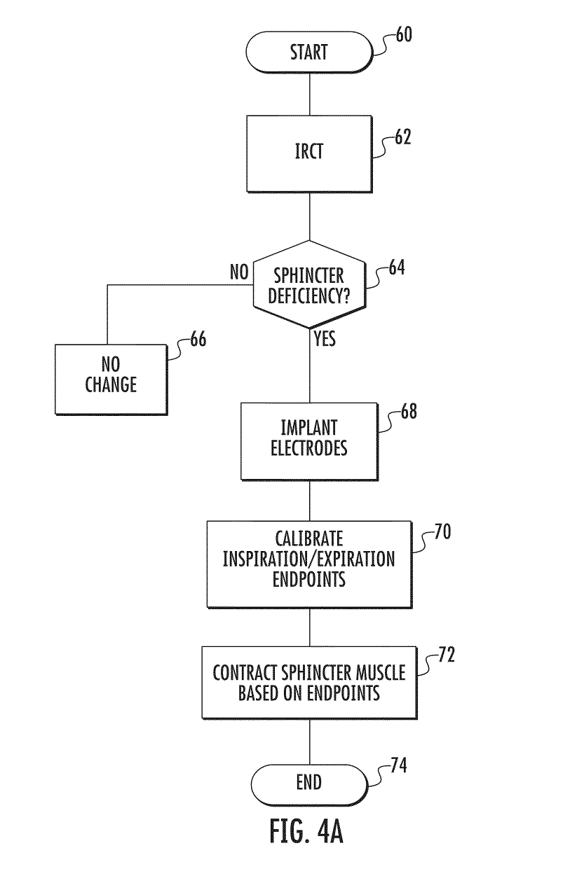

FIG. 4 is a medial view of the female pelvis 40. In this case the transducer as sensors 32a, 32b operate wirelessly and transmit wireless signals at the onset of inspiration, and simulate the electrodes 42, 44 and/or mechanical or electronic IUS or IAS devices (indicated by the multiple dotted lines 42, 44 on the appropriate sphincter). The attached lines represent that the electrodes could be used or other electronic or mechanical devices to close these sphincters. The electrodes 42, 44 close these sphincters during inspiration. The electrodes 42, 44 are implanted in or around the internal urethral sphincter (IUS) 46 and internal anal sphincter (IAS) 48. The sensor(s) 32a, 32b in this example are connected to the sphincter closure devices and/or either directly (via microwire) or indirectly (wireless, radio frequency, Blue-Ray, or similar technology). These pelvic devices as electrodes 42, 44 or other device may close the IUS or IAS either electronically (muscle simulation) or mechanically such as by pressing inward on the sphincter with or without an intervening processor or control unit.

Displacement of the diaphragm may be detected by one or two sensors 32a, 32b or other type of transducers, which are implanted at or on the medial costal border of the eighth rib using a trochar device to implant the small, cylindrical transducers. These motion sensors 32a, 32b as transducers detect the movement of the lower rib cage during deep inspiration. This movement of the rib cage during inspiration occurs as a result of contraction of the diaphragm and the corresponding expansion of the thoracic cavity and abdominal cavity. During inspiration, the costal margins of ribs 8-10 move supero-laterally and the two motion sensors 32a, 32b as illustrated in FIG. 4 may detect an increase in distance between the two devices and thereby rib cage expansion corresponding to inspiration.

It is possible to calibrate the system using a remote control device or computer 52 that is linked to the control unit 34 and operative as a transmitter/receiver and may be used to set the inspiration/expiration endpoints of the rib cage movement via transmission of signals wireless in this example using a transmitter/receiver circuit 52a in the remote device 52. This calibration is performed in the clinical setting by a clinician. The clinician will ask the patient to completely exhale and will then press a [set] button 52b on the remote device 52 at the end of complete exhalation. The motion sensors 32a, 32b may be directly or indirectly connected to the devices 42, 44 as electrodes in this example that close the IUS and/or IAS through muscle contraction, which will control intrinsic sphincter closure based on deep inspiration and the associated inspiration closure reflex as a normal neurological event linked to significant inspiration.

The clinician will ask the patient to deeply inhale (inspiration) and then press the [set] button 52b at the end of deep inspiration. The remote device computer 52 is linked by radio frequency, Blue-Ray or other similar communications link such as to the control unit 34 or directly to the sensors 32a, 32b and will record the inspiration/expiration endpoints and the associated range of rib cage movement. The mode of transmission of signals from the motion transducers 32a, 32b is transmitted by, but not limited to, direct or indirect communication connections to the implanted control unit 34, which can also act as a communications receiver for signals from the sensors 32a, 32b, and through a transmitter function, initiate one or more devices that cause: (1) electronic simulation of the IUS and/or IAS smooth muscle, which will contract these smooth muscle sphincters and prevent voiding and/or evacuation through electronic means; or (2) mechanical closure of the IUS and/or IAS, which contract these smooth muscle sphincters and prevent voiding and/or evacuation; through mechanical means. There can be direct or indirect connection to a mechanical and/or electronic devices, which will close these sphincters through electronic or mechanical means. The closure of these sphincters by these devices may be synchronized with the inspiration closure reflex (ICR), a normal neurological event, which occurs with deep inspiration and thereby increases intrinsic sphincter tonicity prior to a potential increase in intra-abdominal pressure (IAP).

The control unit 34, which is usually implanted, will detect the start of inspiration through the sensors 32a, 32b and initiate corresponding simulation of the microelectrodes or other devices 42, 44 at the internal urethral sphincter (IUS) 46 and/or internal anal sphincter (IAS) 48 and thereby increase sphincter tonicity. The device operates through its communications circuitry 35. During urinary voiding or evacuation of the bowel, the control unit 34 may be temporarily turned off and permit volitional voiding and/or evacuation of the urinary bladder or bowel, respectively. Pressing an ‘on’ button again, resets the device to the previous setting for respiration and control of sphincter tone and allows synchronizing of the devices or electrodes 42, 44 with the patient's inspiration.

The description relative to FIGS. 1-4 assist in understanding the ICR and use of the sensors as transducers 32a, 32b for diagnosing physiological conditions. The system will not usually detect phrenic nerve activity and will not usually use devices to stimulate the inferior hypogastric plexus, which innervates, in part, the internal urethral and internal anal sphincters. That type of device would involve a more invasive surgical procedure and it may be more difficult to control the IUS and IAS as the plexus innervates other pelvic structures.

The process shown relative to FIGS. 1-4 starts (block 60). The involuntary reflex cough test is administered (block 62). A determination is made if there is sphincter dysfunction (block 64). If not, then there are no changes made (block 66). If yes, then electrodes are implanted (block 68). The inspiration/expiration endpoints are then calibrated (block 70). Sphincters are contracted based on the endpoints during inspiration and expiration (block 72). This process continues until a determination is made that sphincters no longer need to be contracted and the process ends (block 74). It would be rare to have the process end since usually a patient will require the treatment over a long period of time.

Detection of diaphragmatic contraction is anatomically complicated by its close proximity to adjacent structures. Any implants or electrodes in the diaphragm may damage or injure these structures, e.g., heart, lungs, gastrointestinal tract, abdominal organs, etc., or cause a pneumothorax, hemothorax or similar breech of the pleural cavity. Thus, such a device may not be desirable. The system usually will not use a device to detect electrical activity of the phrenic nerve.

This type of system is more reliable than a phrenic nerve stimulator. If neuropathy is the cause of the ICR breakdown, it is possible to assume all nerves have some degree of ongoing neuropathy, which may get worse. If the phrenic nerve fails to activate the diaphragm, thus causing shorter movements, it may be assumed that the same process occurs with the Inferior Hypogastric Plexus to the ICR. It is possible to override these deficits to the ICR and reset the closure variables, adjustable over time, using a more reliable method than nerve assessment or activation.

The phrenic and diaphragm may be adequate but the lumbosacral stenosis injures the nerves that close the IUS. Any closure settings by this detection would be different compared to the phrenic nerve and diaphragm function. IUS activation is based in this instance on the present ability to activate the diaphragm, reflected by rib movements. If a subject is restricted in inspiration, COPD, arthritis, kyphosis or restrictive patterns of breathing, the system resets the IUS closure sensitivity to less activation from the ribs. These settings may be individually customized by the Urologist and may occur in many different patterns. eased on the ICR deficit, they are adjustable by the urologist in the clinic or with an urodynamics examination. The adjustment may be compared to other adjustment technologies, e.g., insulin, pain medicine or intrathecal Baclofen pumps. There is an override to void if the subject cannot relax and possibly deactivate a sensitive ICR setting, similar to a restrictive, kyphotic type patient. Many other options are possible.

Tunneling for the microwires 50 to the electrodes 42, 44 is straightforward to the level of the pubis. Connecting a microwire 50 to an electrode 42, 44 (or electrode array arranged on a tape), however, may require another step. It is possible to use a curved trochar or instrument similar to that used for a supra-pubic urinary bladder suspension. The wire is connected to the tape when electrodes are contained on the tape and pulled into place by palpating the placement per vagina. FIG. 4 shows a tape 58 in block format that supports the illustrated electrode array 44. It should be understood that the electrodes could be single electrodes, an array of electrodes such as on a tape or other support or other configuration.

The electrodes 42, 44 adjacent to the IUS and IAS may require a power supply and there may not be room for the power supply in the area of the pelvis, but there are improvements in power supply, especially since MEMS technology may be used for sensors and power supplies. The electrodes 42, 44 as stimulators are small and do not migrate. Another consideration for design and placement is the vascular layout of vessels and pathway of nerves, which in this area may be problematic.

It may be possible to use sensors 32a, 32b that are programmed to work with each other and the transponder devices such as the electrodes 42, 44 by movement changes. It is possible to activate the electrodes 42, 44 without wire placements. Electrodes may also be activated by sensors directly attached to them so that there are no wires and the sensors/electrodes are formed as integrated units. Possible communication linkages include Bluetooth or similar wireless technology to activate the electrodes from the control unit 34.

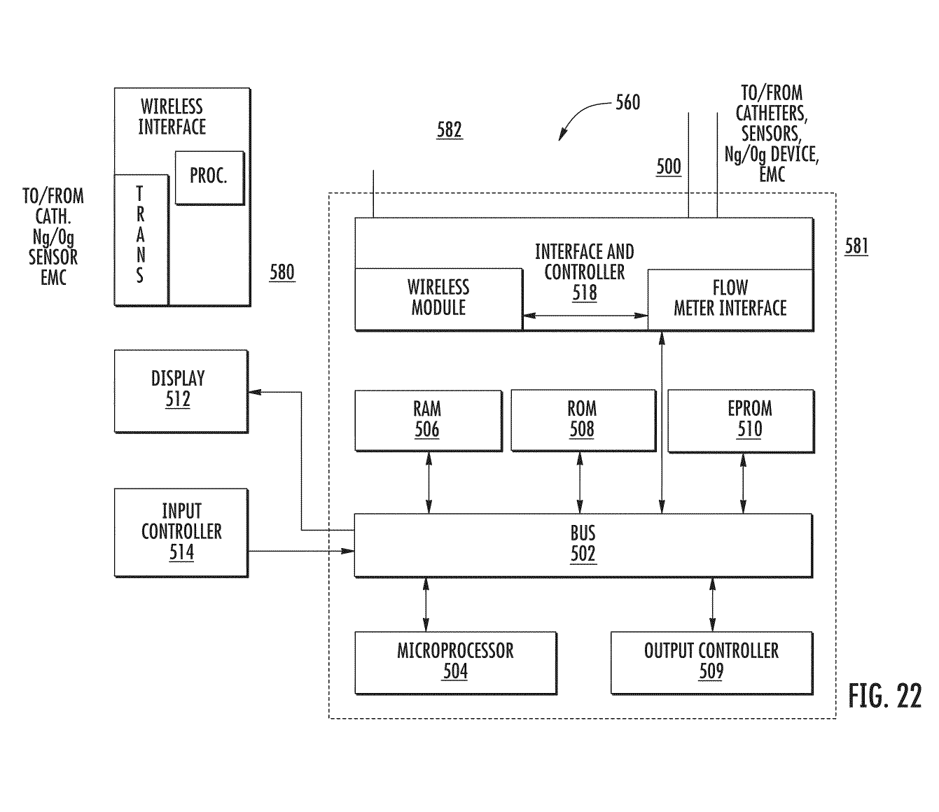

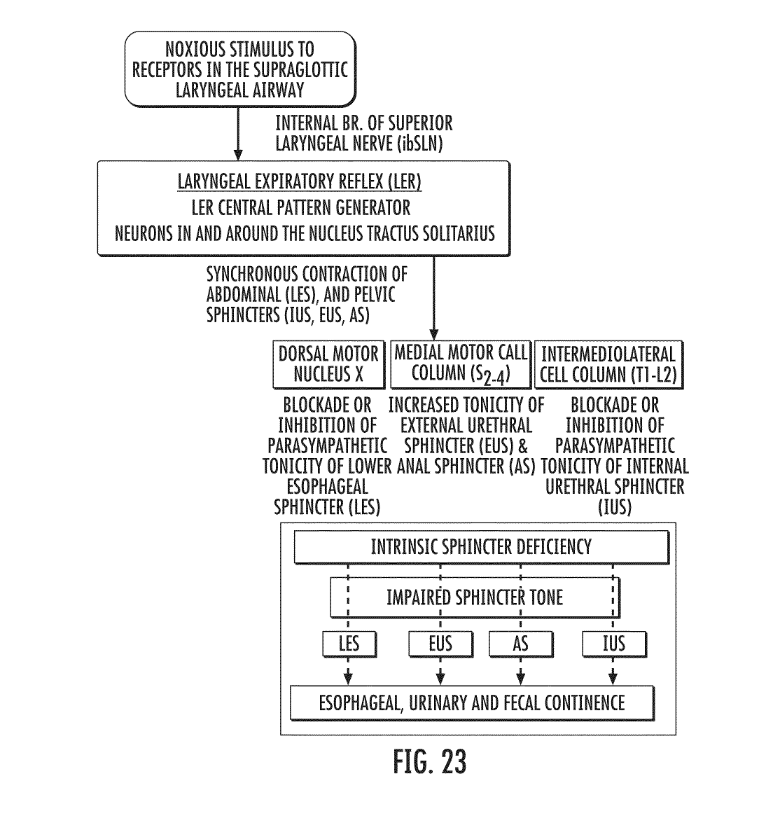

IRCT (involuntary reflex cough test) testing will provide more reliable data sampling of extubation failure risk than VC (voluntary cough) and especially tracheostomies, which are the majority of prolonged intubated patients. There are many variables to the possible scenarios and they require clinical judgment. It is possible to add a micro tube with a transducer that can plug into a processing device, such as the handheld processing device shown in FIGS. 21 and 22 and also Ng/Og or urology tubes as disclosed in commonly assigned patent application Ser. Nos. 12/878,257 filed Sep. 9, 2010; Ser. No. 12/878,281 filed Sep. 9, 2010; and Ser. No. 12/878,316 filed Sep. 9, 2010, the disclosures which are hereby incorporated by reference in their entirety. These devices can be inserted via a percutaneous endoscopic gastrostomy tube (PEG) or Jejunostomy (J-tube) and used to calculate the iRCT cough epoch values for IAP responses to help determine extubation risk.

Many prolonged intubated patients are converted from Ng/Og tubes to PEGS or J tubes for feeding, and many intubated patients are changed to tracheostomy tubes if it is a prolonged illness. Doctors continually attempt to determine what variables are required to extubate from the larynx or decannulate safely from the larynx with the least risk of reintubation. Some patents require this for post operative pneumonia prevention. Patients that receive tracheostomies are usually sicker, weaker and have a higher risk of decannulating. If it fails, the patient is not in a good place with airway management, and stomas close quickly. Many tracheostomies are usually accompanied by tube feeding from Ng, PEG or J-tubes. The doctor or the clinician may have these tubes with pressure sensors for measurement already in place with the ability to plug into a processing device to measure, or have the ability to insert a transducer to measure through these tubes, which can be removed.

It should be understood that function of the lower esophageal (LES) and internal urethral (IUS) sphincters has not been reported during voluntary and involuntary respiratory maneuvers. As noted before, prospective, barium videofluoroscopy study (BSV) of the LES was performed on four healthy adult males during voluntary cough (VC), laryngeal expiration reflex (LER), breath hold maneuvers and normal inspiration. One subject had fiberoptic pressure catheters placed in the LES and IUS, and EMG recording of the right T7-8 intercostals during respiration.

The BSV showed closure and relaxation of the LES corresponding to the inspiration and expiration of VC. The LES was patent during the LER. There was closure of the LES during the deep inspiration/breath hold event. Pressure catheters in the LES and IUS showed increased pressure during inspiration. These observations suggest that pulmonary inspiration afferents elicit a patterned reflex motor response in the LES and IUS, referred to as the Inspiration Closure Reflex (ICR).

The respiratory cycle is modified in many ways and by many influences that also activate the expiratory muscles for respiration. When the lung was distended by inspiration, pulmonary afferent impulses were conveyed to the brainstem via the Vagus nerve, and these afferent impulses reflexively initiated expiration. When the lung was deflated, other pulmonary afferent receptors were stimulated, and their impulses, also conveyed to the brainstem by the Vagus nerve, reflexively initiated the next inspiration.

Voluntary cough (VC) and the laryngeal expiration reflex (LER) as an involuntary cough have been used for assessment of stress urinary incontinence (SUI) in women and neurological airway protection in humans. The urodynamic tracings from SUI clinical trials suggest that the inspiration during VC stimulates pulmonary afferent fibers that may directly activate closure of the internal urethral sphincter (IUS).

Commonly assigned U.S. application Ser. No. 13/354,100 filed Jan. 19, 2012 by the same inventors, the disclosure which is hereby incorporated by reference in its entirety, discloses a system and method of diagnosing acid reflux using an involuntary reflex cough test. In one example as disclosed, a nasogastric/orogastric (Ng/Og) device is inserted into the stomach and the involuntary reflex cough epoch induced. The intra-abdominal pressure and elevational reflux along the Ng/Og device is measured. In an example, the functional status of the gastric valve is determined based on the measured intra-abdominal pressure and elevational reflux along the catheter.

Use of the involuntary reflex cough test with or without a voluntary cough test is also disclosed in commonly assigned U.S. patent application Ser. Nos. 11/608,316 filed Dec. 8, 2006; 11/550,125 filed Oct. 17, 2006; 12/643,134 filed Dec. 21, 2009; 12/643,251 filed Dec. 21, 2009; 12/878,257 filed Sep. 9, 2010; 12/878,281 filed Sep. 9, 2010; and 12/878,316 filed Sep. 9, 2010; the disclosures which are all hereby incorporated by reference in their entirety. The '257, '281 and '316 applications disclose oral-esophageal-gastric devices, some with esophageal cuffs and/or reflux measurement systems that can be used to assess GERD or determine stress urinary incontinence in some examples using the involuntary reflex cough tests alone or in combination with the voluntary cough test.

There now follows a discussion of materials and testing method. The test included a prospective, barium swallow videofluoroscopy (BSV) study. Four normal, healthy male subjects participated in the BSV study. One of the subjects also underwent evaluation of the IUS and LES using fiberoptic pressure catheters. After review of the study protocol, informed consent was obtained from all subjects. BSV studies of the LES were performed using only thin barium solution in each subject. The subjects were standing for all BSV test maneuvers using a standing anterior-posterior view. Videofluoroscopic photomontages were captured at three second intervals and analyzed for each maneuver.

For the VC, each subject swallowed a small cup of thin barium solution followed immediately by a deep inspiration and a VC. The BSV captured, at the level of the LES, a photomontage of the barium flow during the VC.

The breath hold maneuver required the subject to perform a deep inspiration and breath hold followed immediately by swallowing a small cup of thin barium solution. The BSV captured, at the level of the LES, a photomontage of the barium flow during the breath hold voluntary maneuver. All of the photomontages were visually analyzed to determine the relationship of the barium to the position of the LES and diaphragm.

The induced reflex cough test is a cough provocation test that stimulates the laryngeal expiratory reflex (LER). The LER is a series of expiratory coughs (cough epoch) without a significant preceding inspiration. This LER cough epoch caused 5 coughs (C5) with an average duration of 14.8 seconds. The following materials were used to perform the IRCT: a) vial containing a 20% solution of tartaric acid (Nephron Pharmaceutical, Inc; Orlando, Fla.); b) Pari LCD jet nebulizer (Bonn, Germany); c) oxygen flow meter; d) oxygen tank; and e) gloves and safety mask. The jet nebulizer was FDA approved for use in the U.S. and bore the CE Marking designating the manufacturer's compliance with Council Directive 93/68/EEC.

For the BSV study using the IRCT, the subject swallowed 50 ml. of thin barium solution immediately followed by administration of the IRCT. The BSV captured, at the level of the LES, a photomontage of the flow of barium, during the LER involuntary cough maneuver.

One subject also had both nasogastric and urethral fiberoptic, disposable catheters (#10 and #7 French catheters, respectively) with the pressure sensors placed at the level of the LES and IUS, respectively. Electromyography (EMG) electrodes were placed at the mid-axillary line of the T7-8 intercostal space and were used to confirm the inspiratory activity of the intercostal muscles. The Lumax TS Pro was used to record LES and IUS pressures and EMG activity. All urodynamic (UD) equipment and catheters used in this study were FDA approved for use in the U.S. and bore the CE Marking designating the manufacturer's compliance with Council Directive 93/68/EEC.

The one subject, who participated in the catheter portion of the study, was positioned in a semi-recumbent lithotomy position (approximately 60 degrees head up) such as using the structure shown in FIG. 3B as part of a quantitative analysis of the LES and IUS activity during inspiratory maneuvers. The subject performed deep and shallow breathing and breath hold maneuvers with simultaneous recording of LES and IUS pressures, and EMG intercostal inspiratory activity. The recordings were saved on the Lumax TS Pro for analysis of pressure waves and EMG activity.

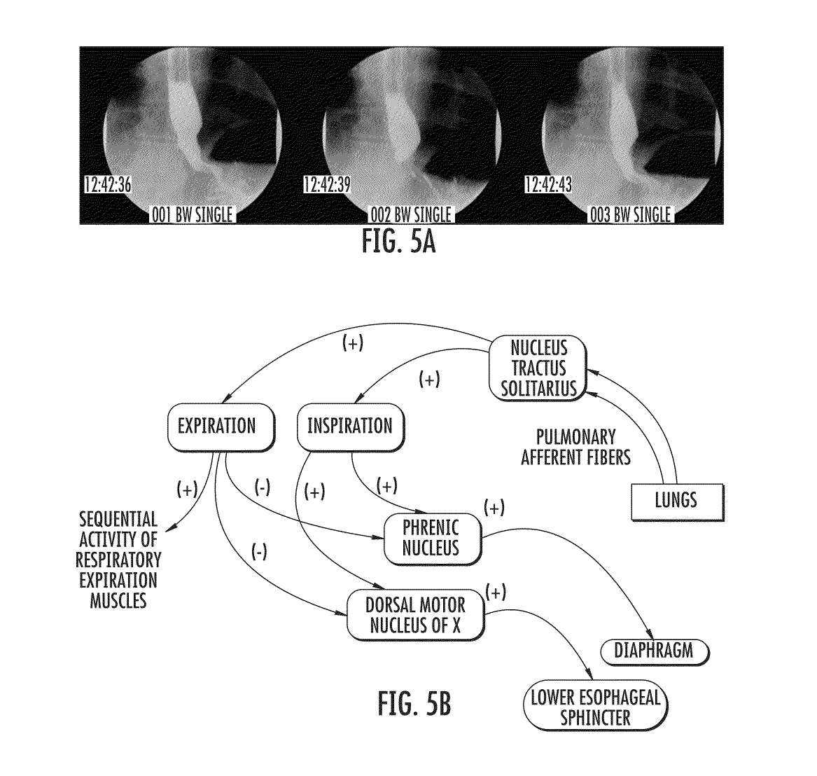

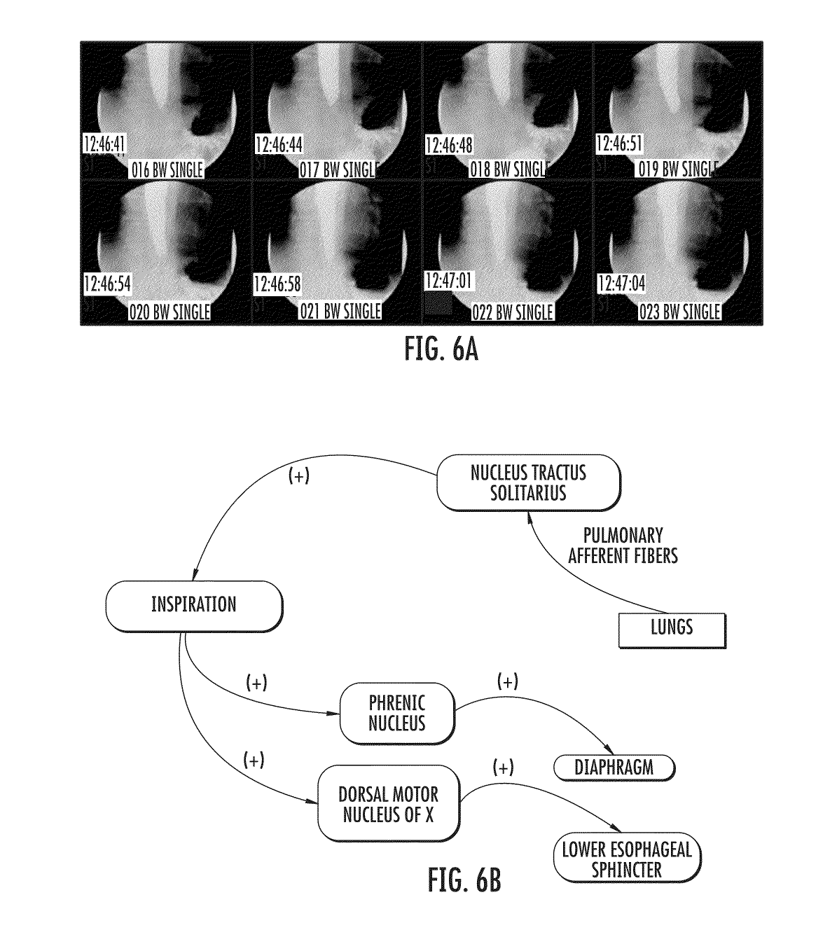

BSV followed immediately by VC showed transient interruption of barium at the LES during inspiration, which released with expiration such as shown in the images in FIG. 5A. FIG. 5B shows a nerve conduction pathway diagram for the inspiration closure reflex. The BSV sequence in FIG. 5A shows the barium swallow (left frame) followed immediately by deep inspiration (middle frame), which closes the LES and stops the flow of barium in the right frame. The expiration during voluntary cough releases the LES and allows barium to enter the stomach. The schematic diagram shows that the inspiration closure reflex (ICR) occurs with the activation of pulmonary inspiratory afferent fibers and their termination in the nucleus tractus solitarius (NTS). Centrally, the NTS influences the activity of the phrenic nucleus, dorsal motor nucleus of X and the sacral autonomic nucleus via descending pathways. This circuit regulates intrinsic sphincter tonicity during inspiration and expiration. This result was reproducible in all subjects.

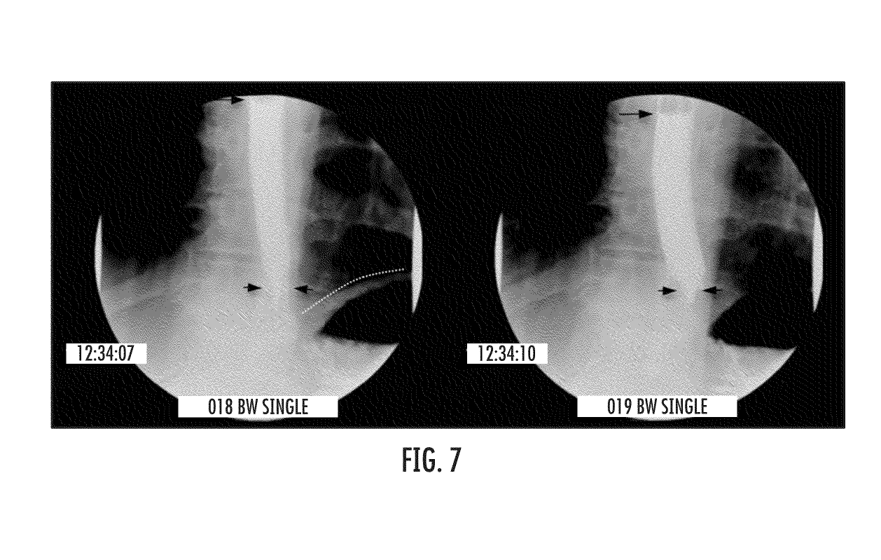

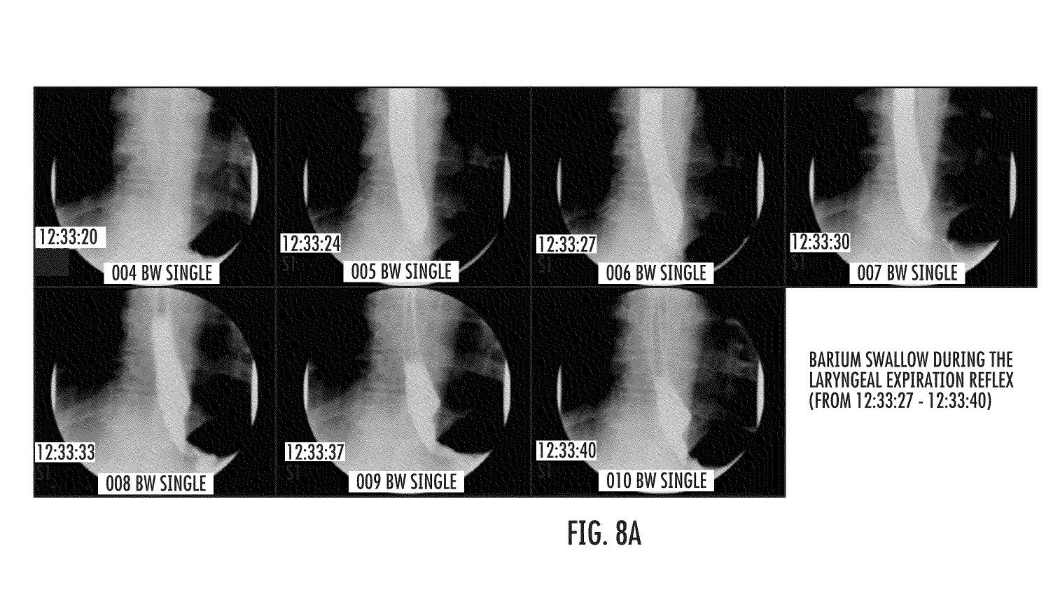

Deep inspiration and breath hold immediately followed by BSV showed complete interruption of barium at the LES during the entire breath hold event as shown in FIGS. 6A and 6B. The photomontage or images in FIG. 6A lasted 23 seconds, and the flow of the barium was completely interrupted at the level of the LES during this entire voluntary maneuver. This result was reproduced in all subjects.

FIG. 6B shows a nerve conduction circuit diagram for a barium swallow during the breath-hold. The ICR is demonstrated in the BSV photomontage images in FIG. 6A and the nerve conduction circuit diagram in FIG. 6B. The images depict the inspiration followed by swallowing barium. The LES closed with deep inspiration and remains closed during the entire duration of breath hold (greater than 20 seconds), which appeared to hold the barium above the LES. The barium stayed above the LES until expiration.

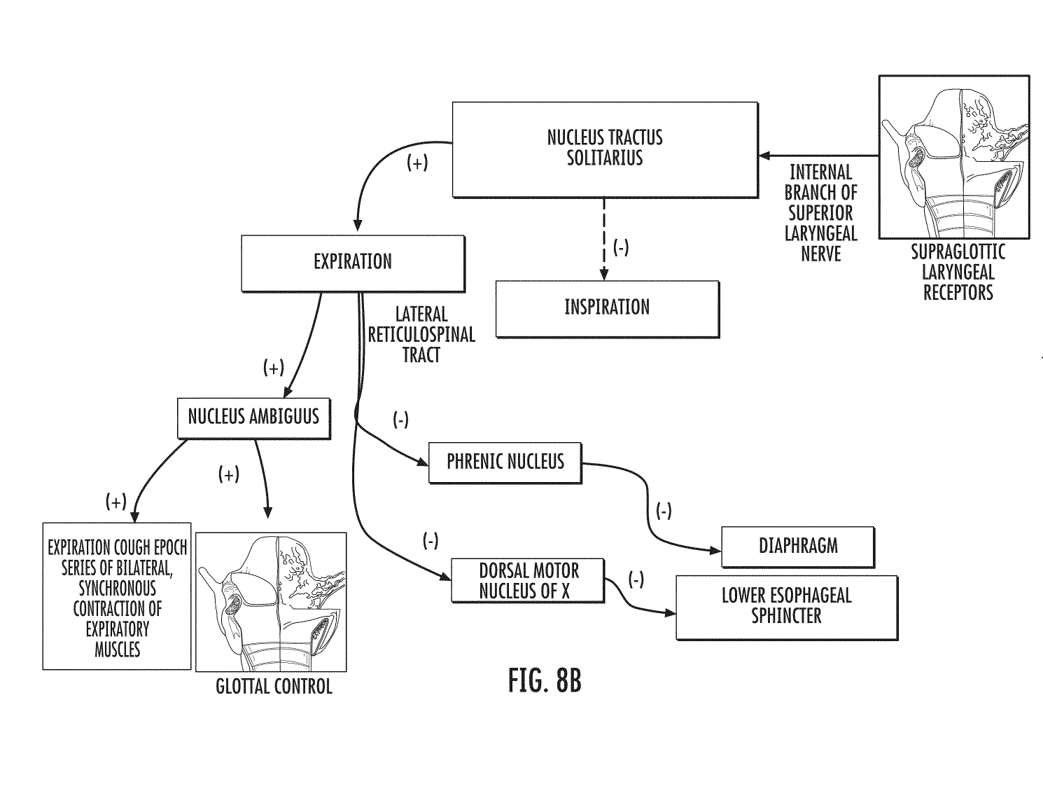

In FIG. 7, at the region of the distal esophagus the diaphragm and proximal stomach were magnified using two consecutive images from a breath hold images as photomontages, which are separated by three seconds. The arrows at the proximal esophagus indicate the level of barium solution. The arrowheads indicate the level of the proximal portion of the LES and the barium solution. The barium in the distal esophagus showed a distinctive V-shaped tapering of the esophagus that suggests a cuff-like closure of the LES. The dotted line in FIG. 7 in the first image was placed above the diaphragm shadow, which was clearly inferior to the distal tip of the barium solution. This result was reproducible in all subjects.

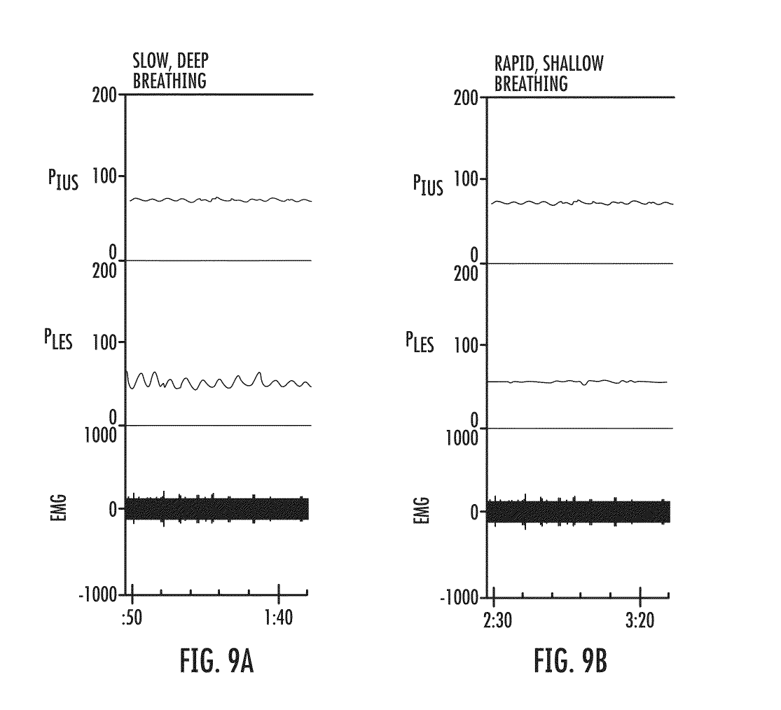

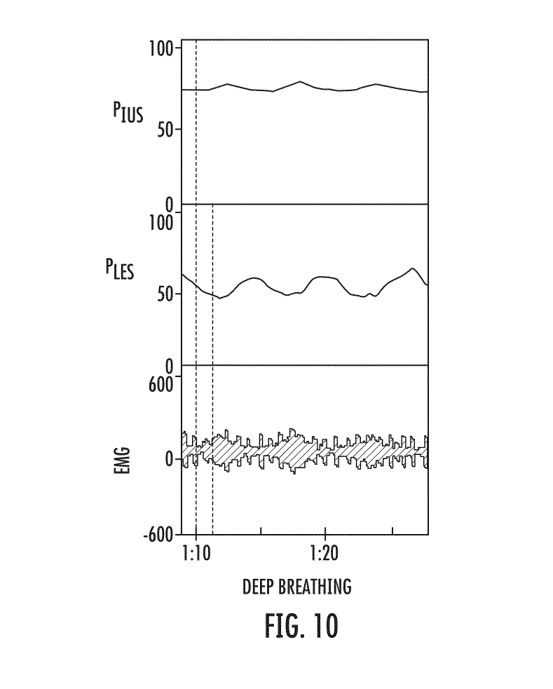

BSV followed immediately by LER activation, using the IRCT, showed no interruption of barium at the LES during expiratory coughs as shown in FIG. 8A. The LER images as photomontages had a 13-second duration without an inspiration. The failure of the LES to close during the LER cough epoch with continuous barium flow was reproducible in all subjects.

FIGS. 8A and 8B show the Laryngeal Expiratory Reflex (LER). The BSV photomontage in FIG. 8A was taken during an LER cough epoch and showed no closure of the LES. The LES appeared to be patent during the LER cough epoch, which allowed barium to flow into stomach. The primary function of the LER is to clear the upper airway when food or fluids have entered the laryngeal vestibule. The nerve conduction circuit diagram in FIG. 8B shows that stimulation of laryngeal receptors, using the IRCT, initiates a series of 5 expiratory “coughs” without inspiration, i.e., the LER cough epoch. The nucleus tractus solitarius influences the phrenic nucleus and dorsal motor nucleus of X, which innervate the diaphragm and LES, respectively. During an LER cough epoch, the LES is patent and inspiration does not normally occur.

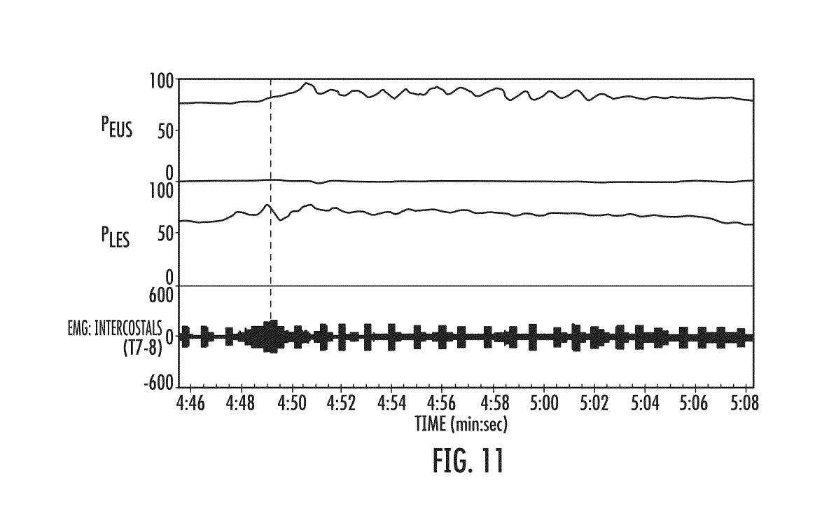

In the subject, who participated in the catheter portion of the study, the rapid closure and pressure elevation of the IUS after the initiation of each inspiration is shown in FIGS. 9B and 9B, which shows pressure recordings of the IUS and LES synchronized with respiration. Simultaneous pressure recordings of the IUS (P_{IUS}) and LES (P_{LES}) with respiratory EMG of the intercostal muscles at the T7-8 interspace demonstrated the activity of the ICR during breathing. During slow, deep breathing and rapid, shallow breathing, pressure waves indicated the respiratory rate and depth dependent variation.

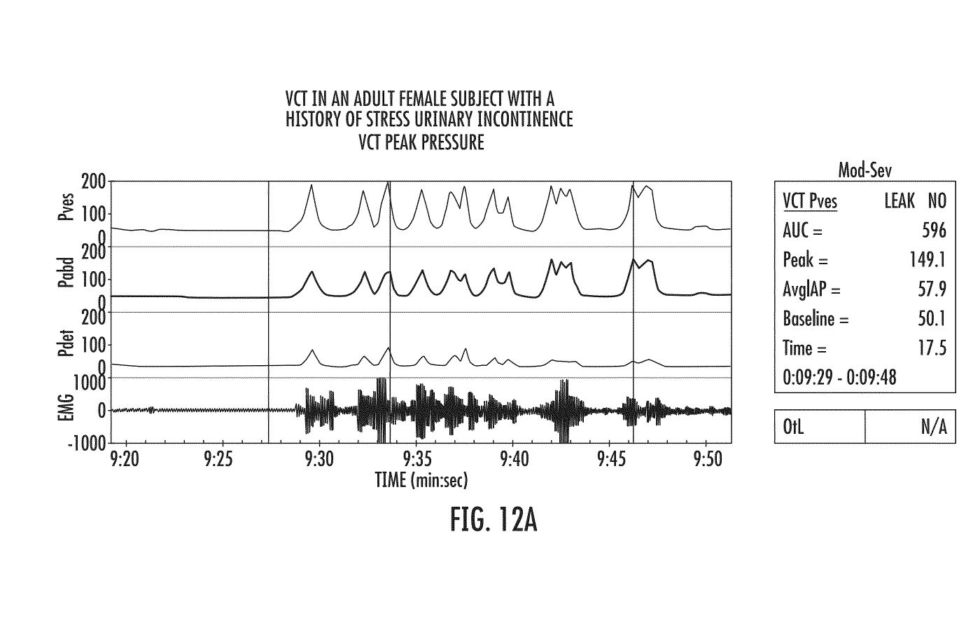

FIG. 10 is a graph showing the latencies of the LES and IUS in relation to inspiration. The closure and pressure elevation of the IUS (P_{IUS}) and LES (P_{LES}) occurred after the initiation of inspiration. These closures (pressure waves) occur before the peak EMG activity, which is before the elevated IAP event in a voluntary respiratory maneuver.

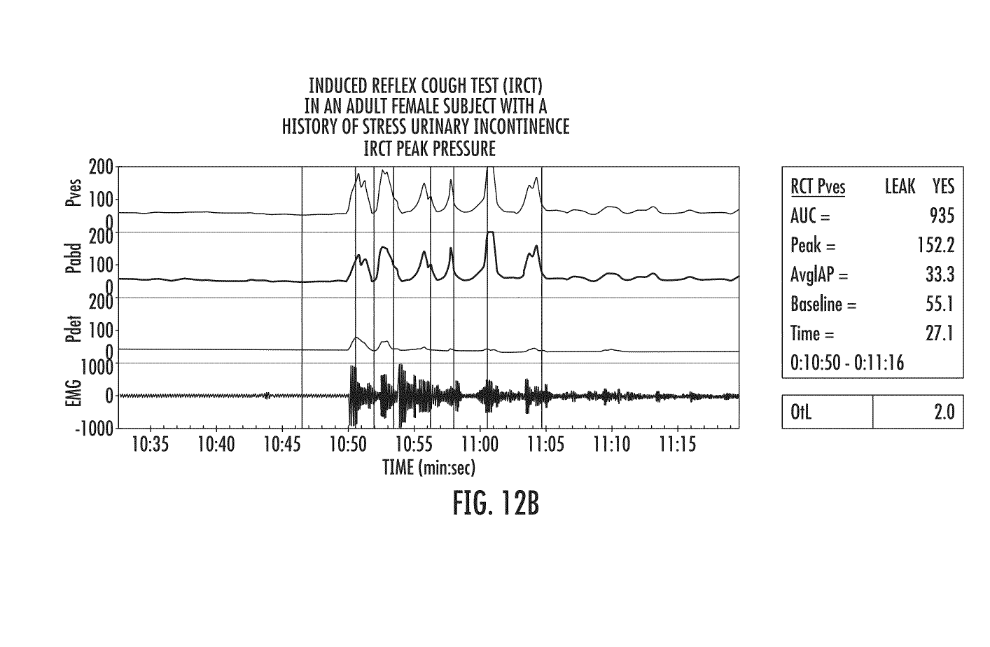

Breath hold caused sustained pressure elevation in the LES (P_{LES}) and IUS (P_{IUS}) and corresponded to overlying voluntary contractions of the external urethral sphincter EUS (P_{EUS}) and pelvic floor musculature as shown in the graph of FIG. 11. During contractions of the EUS and pelvic floor muscles, the pressure of the LES (P_{LES}) remained relatively unchanged. There were no adverse events during this study. The graph in FIG. 11 shows breath hold with maintained pressure elevation in the LES and IUS. Breath hold caused sustained pressure elevation in the LES (P_{LES}) and IUS (P_{IUS}) and corresponded to overlying voluntary contractions of the external urethral sphincter EUS (P_{EUS}) and pelvic floor musculature. During contractions of the EUS and pelvic floor muscles, the P_{LES} remained relatively unchanged.

Respiratory physiology. Bishop further identified expiratory muscle activation as an extension and component of the Breuer reflex. The studies described above suggest that the respiratory maneuvers for control of the closure and pressure of the IUS and LES during inspiration and release with expiration appear to be coordinated and synchronized with the rate and depth of inspiration. This is referred to as the Inspiration Closure Reflex as shown in FIGS. 5A and 5B.

In FIG. 8A, the BSV images as photomontages were taken during an LER cough epoch. The LES appeared to be patent during the LER cough epoch, which allowed barium to flow into stomach. The primary function of the LER is to clear the upper airway when food or fluids have entered the laryngeal vestibule. The LER appears to be inhibitory for inspiration and breathing and the associated reflex motor activations, which prevent closure of the LES during the involuntary cough epoch. Prevention of closure of the LES, during involuntary elevated IAP, may cause reflux of stomach contents in the presence of an incompetent gastric valve.

During slow, deep breathing and rapid, shallow breathing, pressure waves indicated the respiratory rate and depth dependent variation (FIGS. 9A and 9B). Simultaneous pressure recordings of the IUS (P_{IUS}) and LES (P_{LES}) with respiratory EMG of the intercostal muscles at the T7-8 interspace showed the influence of the Inspiration Closure Reflex (ICR). The amplitude of the catheter pressure waves was limited by the sensitivity of the fiberoptic transducers.

FIG. 10 shows an unexpected rapid closure and pressure elevation of the IUS (P_{IUS}) within one second, after the initiation of each inspiration. This delay may be explained by the fast conduction (30-60 m/sec) of the descending pathway in the spinal cord from the nucleus tractus solitarius (NTS) via the lateral reticulospinal tract to the neurons in the sacral autonomic nucleus at S2-4 of the spinal cord. The 25 cm long, unmyelinated, peripheral nerve component conducts at 0.5 m/sec, and takes less than one second to close the IUS. The LES closure was slightly delayed by approximately 1.5 seconds after the initiation of inspiration. This may be due to the different pathway from the NTS to the dorsal motor nucleus of X and a long peripheral, unmyelinated vagal nerve (50 cm) to the LES. Both of these closures (pressure waves) occur before the peak EMG activity, which is before the elevated intra-abdominal pressure (IAP) event in a voluntary respiratory maneuver, e.g., voluntary cough or a Valsalva maneuver.

Control of the LES may be due to upper and lower esophageal reflexes and diaphragmatic reflexes, i.e., a crural reflex. Some studies refer to transient relaxation or inhibition of the LES in association with swallowing obstructive sleep apnea, mechanical ventilation and a negative pressure body ventilator. In previous animal and human studies, respiration pressure “artifacts” in the LES and IUS were not noted or were electronically filtered by manometry instruments. There may be respiratory influences on intrinsic sphincter function that have not been adequately evaluated.

In animal models that require cannulation for respiration and/or positive mechanical ventilation, or in anesthetized animals or humans, the ICR may not have been observed. There has been some description of a “straining crural reflex” during the Valsalva maneuver that caused LES closure by esophageal-diaphragmatic reflexes. In human studies with a negative pressure body ventilator (“iron lung”), pulmonary inspiration afferent fiber activity was abolished during negative pressure inspiration in healthy, non-anesthetized subjects. This type of negative inspiration pressure ventilation and the absence of the subject's initiation of pulmonary inspiration afferent fibers abolished or significantly diminished manometric pressure of the LES during inspiration.

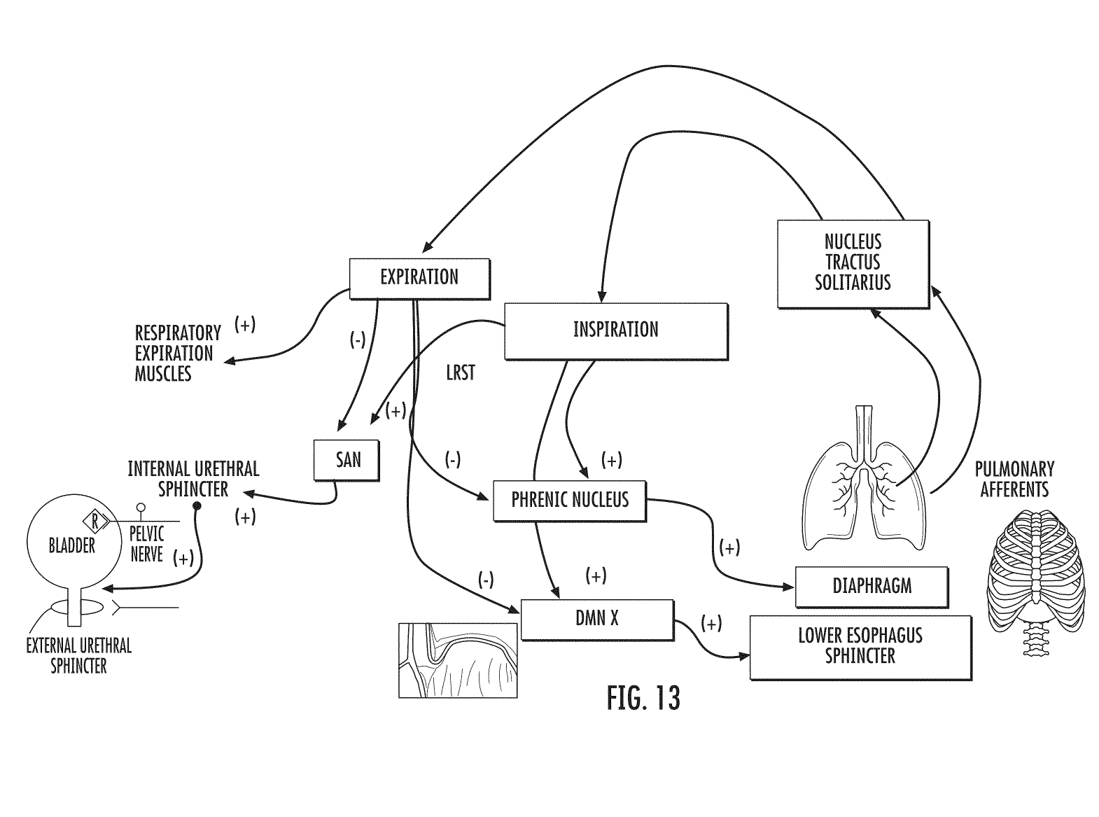

During breath hold, the elevated pressures of the IUS and LES were sustained during the entire breath hold event. The volitional contractions of the EUS and pelvic floor muscles were observed on top of the IUS pressure wave, which were not present on the elevated LES pressure tracing (FIG. 11). A clinical example of the ICR function is shown in the urodynamic (UD) tracing of a series of voluntary and involuntary coughs in a female subject, who has moderate/severe SUI as shown in FIGS. 12A and 12B. The subject had an almost two-fold increase in average IAP with the VC, and each cough was preceded by a deep inspiration (inhalation). During the VC as shown in FIG. 12A, it is believed that the deep inspiration that preceded VC activated the ICR and closed the IUS and resulted in a false negative result for SUI in this “moderate to severe” subject. During the involuntary cough epoch as shown in FIG. 12B, the IRCT UD tracing revealed multiple urinary leaks indicated by the marked vertical lines despite lower average IAP measurements compared to the VC.

These studies on IUS and LES activity, during respiratory events, suggest that if pulmonary inspiration afferent fibers are activated, these intrinsic sphincters close with every inspiration and release with every expiration. During voluntary maneuvers such as VC, Valsalva maneuver, or sneezing, these intrinsic sphincters release tonicity with expiration. The degree of intrinsic sphincter closure appears to vary with the rate, depth or volitional modification of inspiration. The LES and IUS pressure responses seen in this study appear similar to the “respiration artifacts” in other studies. It is possible that the IUS closure and pressure elevation related to inspiration could give a structural advantage at the neck of the urinary bladder to prevent incontinence as shown in FIGS. 12A and 12B. During inspiration, it is also possible that pulmonary inspiratory afferent fibers to the nucleus tractus solitarius (NTS) may co-activate the phrenic nucleus, dorsal motor nucleus of X (DMN) and the sacral autonomic nucleus as shown in FIG. 11. In FIG. 7, the LES closure and pressure elevation via the activation of the DMN may coincide with simultaneous activation of the diaphragm. This simultaneous activation may prevent hiatal herniation during elevated intra-abdominal pressure events such as Valsalva maneuver or pushing during labor and delivery.

There were a small number of subjects in the study, but the findings were method-dependent and reproduced in the four normal, healthy subjects for BSV and the one subject who had both the BSV and catheter studies.

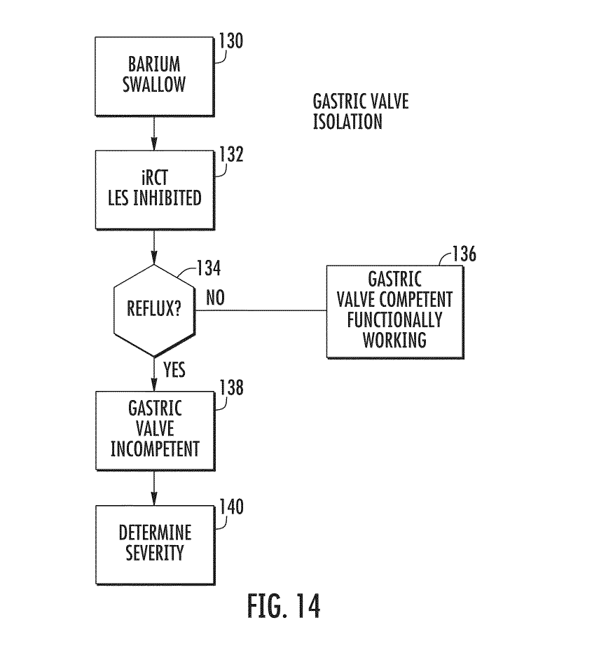

FIG. 13 is another nerve conduction circuit diagram showing the inspiration closure reflex. This diagram shows that the ICR occurs with the activation of pulmonary inspiratory afferent fibers and their termination in the Nucleus Tractus Solitarius (NTS). Centrally, the NTS influences the activity of the phrenic nucleus, dorsal motor nucleus of X and the sacral autonomic nucleus via descending pathways. This nerve conduction pathway circuit regulates intrinsic sphincter tonicity during inspiration and expiration.

As noted before, there is a control unit 34 as shown, for example, in FIGS. 1-4 that is operative to correlate changes in the intra-abdominal pressure and duration with the depth of inspiration and processes the data for direct microwire or indirect wireless transmission to stimulators of the smooth muscle of the internal urethral sphincter or striated muscle for the external urethral sphincter or anal sphincter. Thus, there is either trans-urethral or trans-vaginal implantation of muscle stimulators to the IUS, EUS and/or AS. It is possible to test the smooth muscle using intra-urethral electrodes or a pressure transducer catheter. One technique to test the maximal urethral closure pressure is to take the deepest breath possible. It is thus possible to treat stress incontinence with a stimulator and it is possible to address a deficit and identify and diagnose a deficit in the physiology by doing a maximal urethral closure pressure with inspiration and use an internal urethral sphincter transducer based on urodynamics.

There now follows detail of the disclosure from the incorporated by reference '841 patent application.

Research on the LES and gastric valve indicates that problems arise with the gastric valve and there is a need for an available test to assess the competency of the gastric valve. In accordance with a non-limiting example, the involuntary maneuver, i.e., the involuntary cough test is employed.

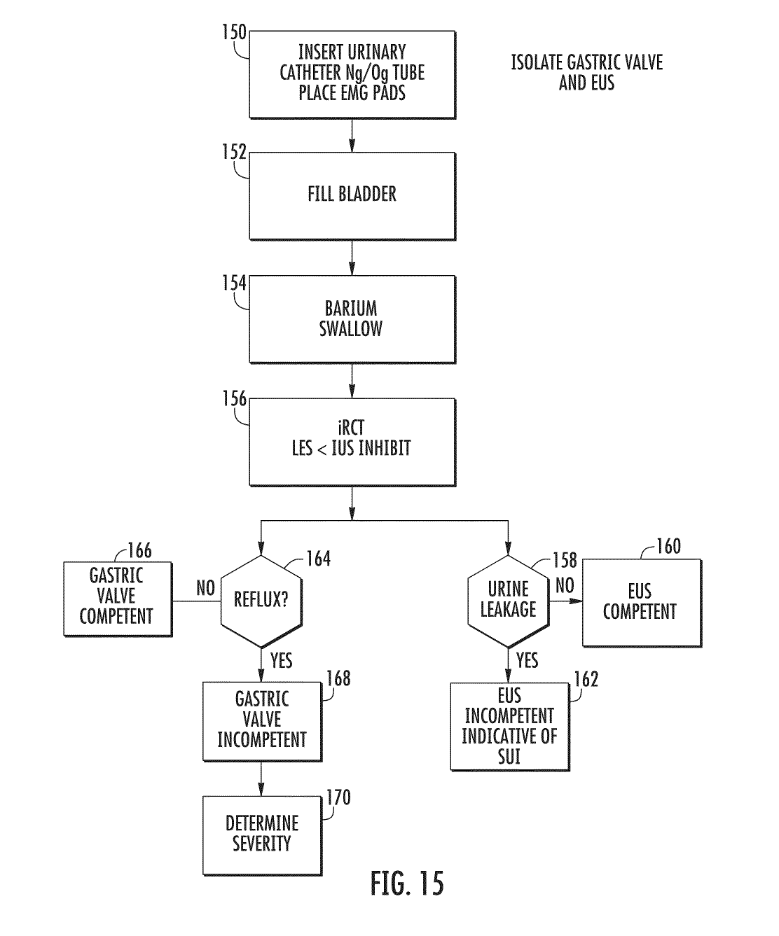

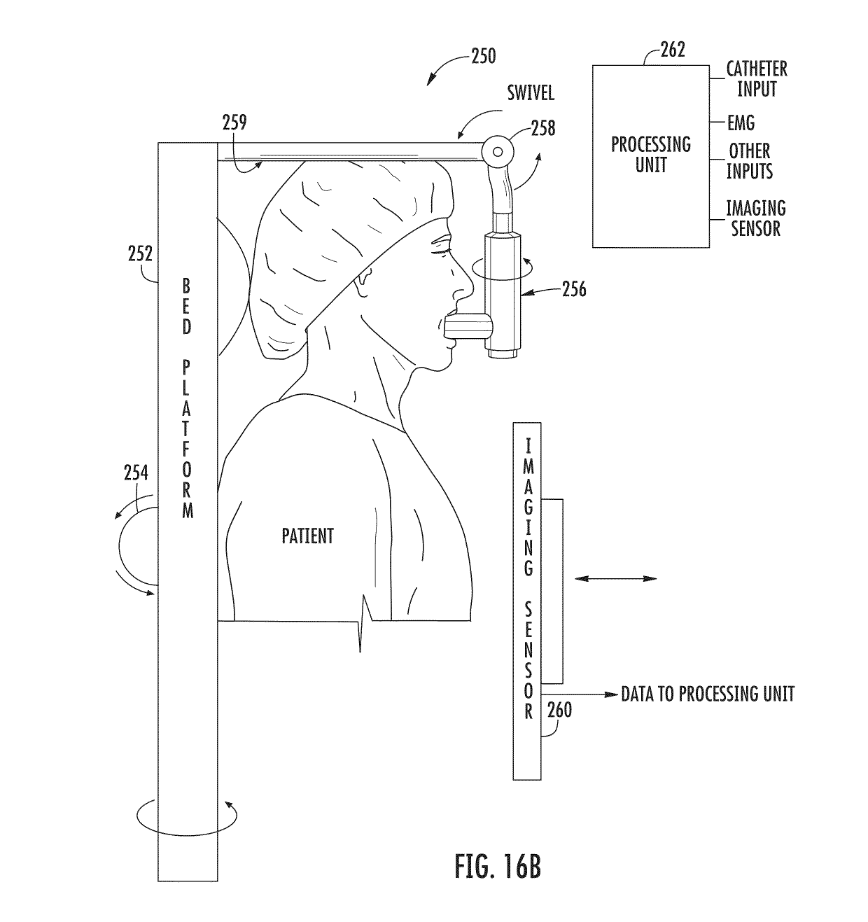

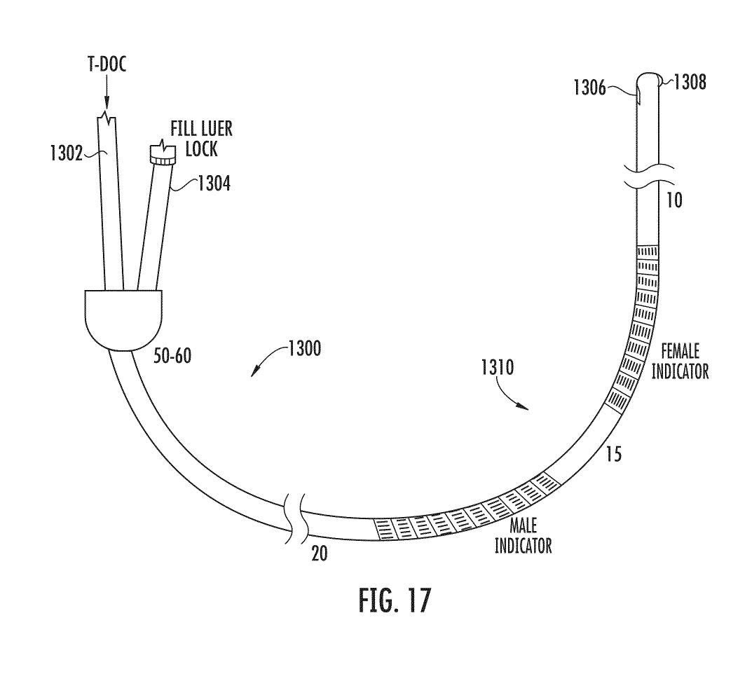

FIG. 14 is a flowchart showing a general sequence of steps that can be used for isolating the gastric valve and determine if the gastric valve is competent and functioning adequately in one example. The kit shown in FIG. 16A can include the components for use with this methodology described relative to FIG. 14 and be used with the test system shown in FIG. 16B as explained below.

The sequence begins with a barium swallow (block 130) immediately followed by the involuntary reflex cough test, i.e., iRCT, such as by inhaling a chemo-irritant such as L-tartrate through a nebulizer in one non-limiting example (block 132). The involuntary reflex cough test isolates the gastric valve from the LES. A determination is made using video fluoroscopy, for example, if the reflux has occurred (block 134). If not, the gastric valve is competent and correctly functioning (block 136). If reflux occurs, then the gastric valve is incompetent and is malfunctioning since it is allowing the reflux (block 138). It is possible to determine the severity of the reflux (block 140), for example, by measuring the amount of reflux that occurs during the involuntary reflex cough epoch to estimate the severity of the malfunctioning gastric valve. This can be accomplished using enhanced fluoroscopy or using a Ng/Og catheter located at the LES or other location as later described to determine the extent of reflux.

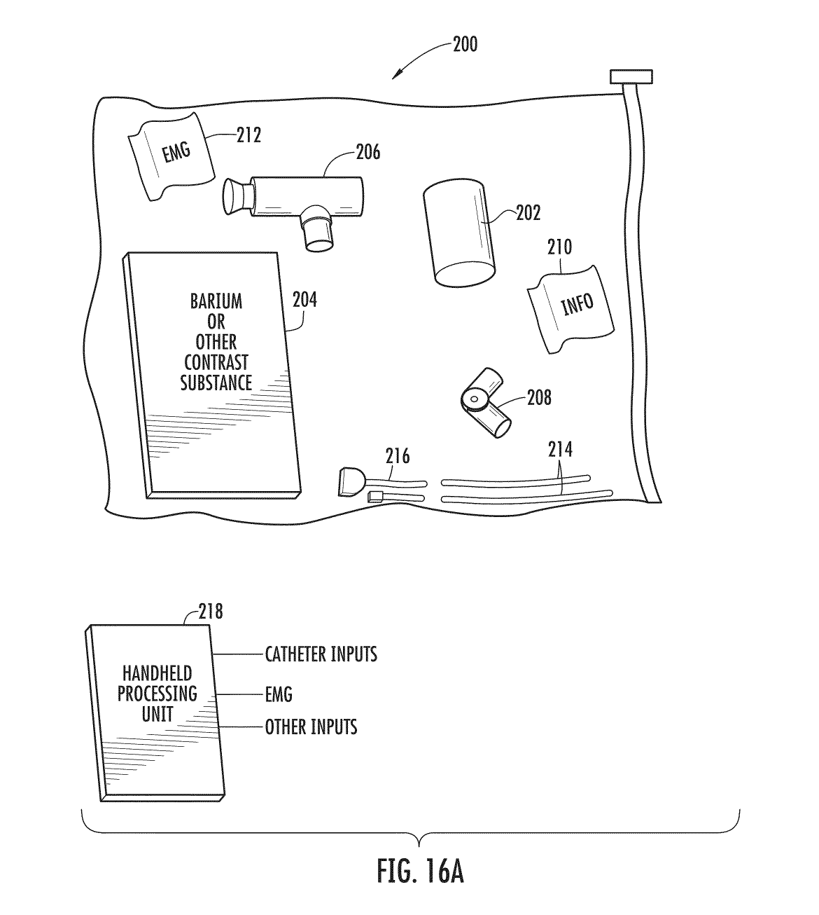

FIG. 15 is another flowchart showing a sequence of steps used for assessing the competency of the gastric valve and isolating the gastric valve from the LES and also isolating the external urethral sphincter from the internal urethral sphincter to determine stress urinary incontinence.

The process begins by inserting a urinary catheter in the patient with a pressure sensor in one example and a sensor located at the internal urethral sphincter in an example. The Ng/Og tube may include at least one sensor to be positioned at the LES and pH sensor at different positions. EMG pads can also be positioned at appropriate locations at the mid-axillary line of the T7-8 internal space (block 150). This could also include the paraspinals. The bladder is filled such as with saline solution (block 152). Barium or other contrast material is swallowed (block 154) and the involuntary reflex cough test induced (block 156). Two analysis paths are shown. A determination is made whether urine leakage occurred (block 158). If not, then the external urethral sphincter is competent and functioning adequately (block 160). If yes, then the external urethral sphincter leaked indicative of stress urinary incontinence (SUI) (block 162). Some determination of the severity of SUI or other problems can possibly be determined through analyzing the EMG results together with any intra-abdominal pressure that has been recorded during the involuntary reflex cough epoch. Reference is also made to the incorporated by reference applications for appropriate data and analysis regarding same. A determination is also made whether reflux occurred (block 164). If not, then the gastric valve is competent and functioning adequately (block 166). If yes, then the gastric valve is incompetent and is not functioning correctly (block 168). By using a Ng/Og tube or advanced imaging of the contrast agent, e.g., Barium Sulfate, it is possible to determine the severity of the reflux (block 170) such as measuring the amount of reflux at the LES and other locations within the esophagus.

A patient kit for assessing the gastric valve in conjunction with fluoroscopy and the EUG can be provided and an example is shown in FIG. 16A at 200. Items in this illustrated kit include:

1) Pneumoflex or USA Flex 20% tartaric acid in 3 ml unit dose vial 202;

2) 1000 ml Barium sulfate USP 204;

3) Ion. Nebulizer or Crossfire Nebulizer 206;

4) Swivel adapter for nebulizer 208;

5) Protocol information sheet 210;

6) EMG pads 212;

7) Ng/Og tube or catheter 214; and

8) Urinary catheter 216.

The purpose of this kit 200 is to simplify the assessment of the gastric valve functioning (and/or external urethral sphincter) using the involuntary maneuver, i.e., involuntary reflex cough test (iRCT) to increase the intra-abdominal pressure to isolate the gastric valve while inhibiting the LES and, in some examples, isolating the external urethral sphincter. Evidence of gastric reflux can be observed directly using video fluoroscopy and evidence of SUI determined by isolating the external urethral sphincter to determine when there is urine leakage.

As shown in FIG. 16A, a handheld processing unit, such as described later relative to FIGS. 21 and 22, can be associated with the kit 200 and includes catheter inputs, EMG and other inputs.

It is well known that the gastric valve allows food to enter the stomach but prohibits reflux of gastric acid into the esophagus. As to the patient kit 200, one aspect is the use of the swivel adapter 208 for the nebulizer such that when the patient is turned over, the nebulizer through use of the swivel adapter can be more readily used by a doctor.

There have been a number of previous tests to distinguish different urinary incontinence problems including: 1) increasing the intra-abdominal pressure using a Valsalva maneuver; 2) having the patient jump up and down; or 3) generating one or more strong voluntary coughs. Through much clinical work, such as described herein and in the copending and incorporated by reference patent applications identified above, it has been determined that the involuntary reflex cough test (iRCT) activates the nucleus ambiguus, as compared to the voluntary reflex cough test.

FIG. 16B shows a patient examining system 250 for imaging any contract agent that can be used to implement the methodology as described. The patient examining system includes a bed 252 supported on a swivel/pivot 254 that is typically motor driven and allows the bed to be rotated and pivoted to place the patient in any predetermined position as inclined or turned over, if necessary. A nebulizer 256 is supported on a swivel adapter 258 and rotatable into various positions. The nebulizer 256 can be removable and could include a separate canister (shown by dotted lines at 215) or have nebulized medicine fed through a support arm 259 associated with the nebulizer and swivel adapter 258. Imaging sensor 260 can be positioned adjacent the patient for imaging barium or other contrast agent the patient has swallowed (or been forcibly administered depending on whether the patient is conscious). The processing unit 262 includes various inputs as described relative to the processing unit 218. The processing unit 262 can be a handheld processing unit or a fixed computer connected to the imaging sensor and various catheters inserted in the patient. The imaging sensor 260 in one example is a fluoroscopic instrument configured to image the contrasting agent. The imaging sensor is typically connected to the bed and moveable into a position adjacent the patient to image the contrast agent as it flows through the esophagus into the stomach during the involuntary reflex cough epoch. Data is transferred to the data processing unit where the data is processed and the amount of reflux that occurs during the involuntary reflex cough epoch measured to estimate the severity of the malfunctioning gastric valve in one example or the extent of the gastric valve adequate functioning. This could be accomplished, for example, by comparing a plurality of photomontages taken by the image sensor during the involuntary reflex cough test.

This application is related to copending patent application entitled, “METHOD FOR TREATING AT LEAST ONE SPHINCTER IN A PATIENT,” which is filed on the same date and by the same assignee and inventors, the disclosure which is hereby incorporated by reference.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

1. A system to diagnose the function of intrinsic sphincters, comprising:
a muscle contraction device located on at least one of the urethral and anal sphincters; and
a controller connected to the muscle contraction device and operative to transmit control signals to the muscle contraction device located on at least one of the urethral and anal sphincters to contract the muscle during an inspiratory phase of respiration.

2. The system according to claim 1, comprising sensors positioned to detect movement of the ribs indicative of the inspiratory phase of respiration.

3. The system according to claim 2, wherein said sensors are positioned using sensors placed at the anterior surface of the medial border of the costal margin of ribs 8, 9 or 10.

4. The system according to claim 1, wherein a muscle contraction device is located on one of the at least internal and external urethral sphincters.

5. The system according to claim 1, wherein said muscle contraction device comprises an electrode to stimulate the muscle using an electrical current.

6. The system according to claim 5, wherein said controller is operative to transmit control signals to the electrodes to initiate electrical stimulation.

7. The system according to claim 1, and further comprising microwires connecting the controller and electrodes.

8. The system according to claim 1, wherein the controller is embedded in the abdominal wall.

9. The system according to claim 1, wherein said muscle contraction device comprises a mechanical mechanism on at least one of the urethral and anal sphincters and configured to close the sphincter.

10. A system to diagnose the function of intrinsic sphincters, comprising:
a muscle contraction device located on at least one of the urethral and anal sphincters; and
a controller connected to the muscle contraction device and operative to transmit control signals to the muscle contraction device located on at least one of the urethral and anal sphincters to contract the muscle during an inspiratory phase of respiration based on inspiration/expiration endpoints of rib cage movement.

11. The system according to claim 10, comprising sensors positioned to detect movement of the ribs indicative of the inspiratory phase of respiration.

12. The system according to claim 11, wherein said sensors are positioned using sensors placed at the anterior surface of the medial border of the costal margin of ribs 8, 9 or 10.

13. The system according to claim 10, wherein said muscle contraction device comprises an electrode to stimulate the muscle using an electrical current.

14. The system according to claim 13, and further comprising microwires connecting the controller and electrodes.

15. The system according to claim 10, wherein said controller is embedded in the abdominal wall.

16. The system according to claim 10, wherein said muscle contraction device comprises a mechanical mechanism on at least one of the urethral and anal sphincters configured to close the sphincter.
